(19) United States
(12) Patent Application Publication
(10) Pub. No.: US 2025/0182858 A1
(43) Pub. Date: Jun. 5, 2025

(54) Large Language Model for Unified Text and Point Cloud Molecular Input

(71) Applicants: Insilico Medicine IP Limited, Hong Kong (HK); Insilico Medicine Al Limited, Abu Dhabi (AE)

(72) Inventors: Aleksandrs Zavoronkovs, Hong Kong (HK); Maksim Kuznetsov, Montréal (CA); Rim Shayakhmetov, Montréal (CA); Daniil Polykovskiy, Montréal (CA)

(21) Appl. No.: 18/909,488

(22) Filed: Oct. 8, 2024

Related U.S. Application Data
(60) Provisional application No. 63/604,657, filed on Nov. 30, 2023.

(51) Int. Cl. G16B 40/30; G06T 7/73; G16B 15/00

(52) U.S. Cl. CPC G16B 40/30; G06T 7/75; G16B 15/00; G06T 2207/10028; G06T 2207/20081; G06T 2207/20084

(57) ABSTRACT

A transformer model architecture is described. The transformer model architecture comprises a point cloud input module, a text input module, a point cloud encoder module operatively coupled with the point cloud input module, a large language model module operatively coupled to the text input module and point cloud encoder module and configured to receive data therefrom, and a text output module operatively coupled to the large language model module. The text output module is configured to output molecular data in line notation format.

### CROSS-REFERENCE TO RELATED APPLICATIONS

This patent application claims priority to U.S. Provisional Application No. 63/604,657 filed Nov. 30, 2023, which provisional is incorporated herein by specific reference in its entirety.

### BACKGROUND

### Field

The present disclosure relates to computer-implemented protocols for generating data related to molecular properties.

### Description of Related Art

Previously, computer-based models have been developed to generate diverse and physically plausible models of three-dimensional (3D) structures. While these computer-based models have proven to be adequate for some tasks, there remains a need for improved computer-based models useful for describing and predicting more complex 3D structures.

Artificial Neural Networks (ANNs) are computing systems inspired by the biological neural networks that constitute animal brains. An ANN is based on a collection of connected units or nodes called artificial neurons, which loosely model the neurons in a biological brain. Each connection, like the synapses in a biological brain, can transmit a signal to other neurons. An artificial neuron receives a signal then processes it and can signal neurons connected to it. The “signal” at a connection is a real number, and the output of each neuron is computed by some non-linear function of the sum of its inputs. The connections are called edges. Neurons and edges typically have a weight that adjusts as learning proceeds. The weight increases or decreases the strength of the signal at a connection. Neurons may have a threshold such that a signal is sent only when the aggregate signal crosses that threshold. Typically, neurons are aggregated into layers. Different layers may perform different transformations on their inputs. Signals travel from the first layer (the input layer) to the last layer (the output layer), possibly after traversing the layers multiple times.

Deep Neural Networks (DNNs) are ANNs with one or more hidden layers. These networks, due to their complex structure and large number of trainable parameters, make it possible to solve problems more efficiently. Autoencoders are a subset of DNNs that learn the hidden representation of objects. Objects can be different mathematically formalized objects, for example—strings, graphs, or pictures. An autoencoder includes two parts—an encoder and a decoder. An encoder is an encoding function that maps an object to a point (e.g., latent point) in a numerical space with a specified dimension. This numerical space is called latent space. A decoder is a decoding function that maps a point in latent space to an object in the object space. For training, these networks use reconstruction loss, a function that penalizes the model for differences between the input (encoder input) and output (decoder output) representations of an object.

Generative models (GM) are a subclass of DNNs that enable the generation of objects. Unlike standard DNNs that predict the properties of objects, these networks are trained in such a way as to generate new objects in the future without input data. These models learn the distribution of objects (e.g., distributional learning) and then try to generate samples from this distribution.

Distributional learning generative models generate random molecules by default. However, sometimes one wants to generate objects that satisfy given properties. This formulation of the problem is called conditional generation.

Transformer models are a subset of DNNs that transform an input sequence to an output sequence. These models can determine the context of input sequential data to generate new data in an output. A transformer model architecture generally comprises an input sequence encoder that outputs a tokenized (e.g., a numerical encoded matrix) representation of an input sequence and a decoder that receives the tokenized representation of the input sequence and iteratively generates the output. In the encoder, a multi-headed self-attention mechanism can enable the transformer model to relate each word in the tokenized input sequence with other tokens/words, and to focus on different tokens/portions of the input sequence as it processes each token by amplifying signals of key tokens/portions. The output of the decoder can be transformed into a sequence representing a prediction.

### SUMMARY

A transformer model architecture is described herein. In an embodiment, a transformer model architecture comprises a point cloud input module and a text input module. The transformer model architecture further comprises a point cloud encoder module operatively coupled to the point cloud input module, and a large language model module operatively coupled to the text input module and point cloud encoder module and configured to receive data therefrom. A text output module is operatively coupled to the large language model module and configured to output molecular data in line notation format. The large language model module and the point cloud encoder module may be combined into a single model.

In some embodiments, the point cloud encoder may aggregate spatial position information from a point cloud, and comprise a graph neural network configured to process relative distances between points of a point cloud.

In some embodiments, the transformer model architecture may comprise a plurality of graph neural network layers, where each of the graph neural network layers aggregates information from connected nodes and edges and processes global information from a whole graph.

In some embodiments, the graph neural network may comprise attention mechanisms to compute attention biases related to edge features and relative positions between points of a point cloud, and update point embeddings based on the computed attention biases.

In some embodiments, the point cloud encoder may be trained in an unsupervised manner using 3D molecular data comprising a point cloud received via the point cloud input module.

In some embodiments, 3D molecular data may comprise, for each point of a point cloud, spatial position data and data representing one or more molecular features. The one or more molecular features may comprise one or more of the following: an atom symbol, an atom charge, an atom name, or a corresponding amino acid name.

In some embodiments, the 3D molecular data may comprise data in at least one of the following chemical language formats: a Simplified Molecular-Input Line Entry System (SMILES) format, a Self-referencing Embedded Strings (SELFIES) format, or a XYZ format.

In some embodiments, the 3D molecular data may represent a large ligand or a protein pocket structure, and may be down sampled based on one or more prioritized points of the point cloud. The one or more prioritized points of the point cloud may comprise at least one of the following: a ligand, an alpha carbon (C-alpha) atom, or a terminal atom of a protein amino acid.

In some embodiments, the point cloud encoder may be configured to infer a point description based on one or more points in a neighborhood of a point; and determine a three-dimensional (3D) position of the point relative to the one or more points in the neighborhood of the point. In some examples, the point and the one or more points in the neighborhood of the point may not be connected by a direct edge.

In an embodiment, a transformer model is provided, the transformer model having a point cloud input module, a text input module, a point cloud encoder module operatively coupled with the point cloud input module, a large language model module operatively coupled with, and configured to receive data from, the text input module and point cloud encoder module, and a text output module configured to output molecular data in line notation format. 3D molecular data comprising a point cloud is fed into the point cloud encoder module in an unsupervised manner. A point description is inferred, by the point cloud encoder module, based on one or more points in a neighborhood of the point. A 3D position of the point is determined relative to the one or more points in the neighborhood of the point. At least some of the one or more points are masked or blurred. At least some of the masked or blurred point features are predicted by the pretrained point cloud encoder module. Random points are sampled, and distances between the random points are predicted by the pretrained point cloud encoder module. In some embodiments, a mask or blur loss value may be determined based on the prediction of the masked or blurred point features. A distance loss value may be determined based on the prediction of the distances between the random points, and a weighted sum of mask or blur loss and distance loss may be minimized based on the mask loss and distance loss values, respectively.

In some embodiments, the point cloud may be encoded into one or more point embeddings, and embeddings of an input text sequence may be prepared. The one or more point embeddings and the input text sequence embeddings may be combined to obtain a fused input, and the fused input may be feed into the point cloud encoder module.

In an embodiment, shape-conditioned generation comprises training a transformer model to recover a molecule of a molecular point cloud from a blurred region of 3D space where the molecule is located, where some parts of a molecular point cloud are blurred and a selected portion of the molecule is unaltered and not blurred. 3D molecular data comprising a point cloud is inputted via a point cloud input module. Text representing a desired molecule is inputted via a text input module. The 3D molecular data and text are processed using the trained point cloud encoder module, and a text representing a molecule is outputted in line notation.

In an embodiment, linker design generation comprises training a transformer model to recover a removed part of a molecule when the transformer model architecture does not receive a spatial description of the removed part of the molecule. Data representing one or more molecules is inputted into the trained transformer model. The trained transformer model is used to generate one or more options for a linker portion of a molecule to replace the removed part of the molecule, and one or more molecules having the generated one or more options are obtained for the linker portion of the molecule.

In an embodiment, a computer system comprises a trained transformer model and one or more non-transitory computer readable media storing instructions that, in response to being executed by one or more processors, can cause the computer system to perform one or more of the above-described operations, including, for example, operating the transformer model architecture or pretraining thereof or implementation thereof for shape-conditioned generation or linker design generation of at least one molecule.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 illustrates an overview diagram of spatial molecular generation tasks in accordance with an embodiment.

FIGS. 2A & 2B, which illustrate functional feature-based and modular component-based block diagrams of a point cloud transformer model architecture in accordance with an embodiment.

FIG. 3 illustrates a block diagram of a point cloud encoder module for a point cloud transformer model in accordance with an embodiment.

FIG. 4 illustrates a process for a point embedding computation in accordance with an embodiment.

FIG. 5 illustrates a diagram of embeddings of edges in accordance with an embodiment.

FIG. 6 illustrates a block diagram of a structure of a graph neural network layer in accordance with an embodiment.

FIG. 7 illustrates a point cloud pretraining objective in accordance with an embodiment.

FIG. 8 illustrates a block diagram of a pretraining process for a point cloud transformer model in accordance with an embodiment.

FIG. 9 illustrates a flowchart for pretraining a transformer model in accordance with an embodiment.

FIG. 10 illustrates training and validation results of model performance on shape-conditioned generation task in accordance with an embodiment.

FIG. 11 illustrates training and validation results of model performance on pocket-conditioned generation task with respect to non-blurred fragments in accordance with an embodiment.

FIG. 12 illustrates examples shape-conditioned generated molecule structures in accordance with an embodiment.

FIG. 13 illustrates a flowchart of shape-conditioned generation utilizing a trained transformer model in accordance with an embodiment.

FIG. 14 illustrates a flowchart of linker design generation utilizing a trained transformer model in accordance with an embodiment.

FIGS. 15A & 15B illustrate examples of molecular structure generation results compared to other models and a ground truth in accordance with an embodiment.

FIG. 16A illustrates examples of molecular structure generation results in linker design tasks in accordance with an embodiment.

FIG. 16B illustrate examples of molecular structure generation results in pocket conditioned generation tasks in accordance with an embodiment.

FIGS. 17A & 17B illustrate examples of molecular structure generation results of the PC transformer model in comparison with the SQUID model in accordance with an embodiment.

FIG. 18 illustrates an example of a computing system that support techniques for implementing a point cloud transformer model in accordance with an embodiment.

FIG. 19 illustrates a visualization of learned relative biases in accordance with an embodiment.

FIG. 20 illustrates t-SNE visualizations of textual atom token embeddings and textual amino acid token embeddings in accordance with an embodiment.

FIG. 21 illustrates a graphical comparison of model performance on shape-conditioned generation in accordance with an embodiment.

FIGS. 22A-22D shows input point cloud formations.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Language Models (LMs) have shown exceptional natural language understanding and performance across diverse natural language tasks, including language translation, question answering, and code generation. LMs also show efficiency as conversational agents and for engaging in meaningful dialogues. For example, Large Language Models (LLM) show overwhelming performance and dominance in natural language tasks, e.g., they can learn meaningful word representations based on textual context and perform tasks such as translating from one language to another and keeping dialog going even when conversation switches between different topics. LLMs also can take an input text query, and optionally images, to produce text answers. Moreover, recent research has demonstrated the capacity of LMs (e.g., the Gill and IDEFICS published models) to integrate various data modalities, including images, video, audio, and point clouds in addition to text. Thus, LMs can be generalized and used as foundational models in a wide variety of applications. The integration of data modalities can be achieved through the incorporation of additional domain-specific encoders and decoders that convert non-text data to hidden embeddings that can be added alongside text token embeddings in model inputs.

Recent studies have demonstrated the ability of LMs in processing specialized chemical languages, such as SMILES and Self-referencing Embedded Strings (SELFIES) representations. These models exhibit proficiency in understanding and manipulating textual representations of chemical data, enabling their application across various tasks. For example, LMs have been utilized for molecular property prediction, molecular generation, and chemical reaction prediction. LLMs have been shown to be able to understand not only natural language but also chemical languages like SMILES (Galactica, MolFormer, ChemFormer, MolT5, T5Chem) and amino acid sequences (ProGen, RGN2). Therefore, models can work with text chemical data as an input (e.g., molecular property prediction), as an output (e.g., molecular generation), or both (e.g., reaction prediction).

While SMILES and SELFIES representations capture the structural attributes of chemical compounds, they are limited in representing spatial features. Accurately determining the precise arrangement of atoms within a compound and their interactions within the surrounding environment are pivotal elements in various drug discovery methodologies. Recent studies have demonstrated that LMs can effectively generate meaningful chemical 3D structures in text format by utilizing specialized formats such as Protein Data Bank (PDB), Crystallographic Information File (CIF), and XYZ, where each line represents atom coordinates, elements, and additional features. While this approach has shown promise, it often suffers from excessive length, e.g., by requiring dozens of tokens to describe a single atom in 3D. While suitable for molecules with a modest number of atoms, these representation formats become redundant and impractical for larger structures with hundreds of atoms, like proteins. Additionally, these formats lack information on atom connectivity, necessitating the use of external software tools (e.g., the Open Babel chemical toolbox software) to determine chemical bonds. In practice, such external software is highly sensitive to the quality of atom positions, and even minor noise in atom coordinates can significantly alter the reconstructed chemical graph or cause the molecule to break into disconnected fragments.

Further, a huge gap exists between structural two-dimensional (2D) and spatial three-dimensional (3D) chemical data. For example, the protein 3D structure prediction problem was one of the most fundamental and difficult computational tasks in the recent development of the AlphaFold 3D protein structure prediction architecture. In most drug discovery tasks, the 3D representation of the protein pocket (i.e., the protein structure with suitable properties for binding a ligand) is an essential component to discover the ligand compounds that can bind and change the activity of the protein (See, e.g., the Molecular Diffusion (MolDiff) and DiffLinker models).

While recent advancements have shown promise for integrating LMs into a drug discovery pipeline, existing models suffer from limitations like reliance on chemical string representations (e.g., SMILES and SELFIES), which lack spatial features vital for drug discovery. Additionally, attempts to translate chemical 3D structures into text format encounter issues such as excessive length and insufficient atom connectivity information.

Thus, there is a need for a model, such as the novel point cloud transformer model described herein, that can work with chemical text data (as an input, output, or both) and spatial (e.g., three-dimensional) data.

The point cloud transformer model (also referred to herein as the “PC transformer” model or the “transformer” model) described herein combines domain-specific encoder and textual representations (tokens) of spatial atom arrangements for generative tasks involving 3D molecular structures, including tasks that require effective handling of spatial atom configurations both as inputs and outputs. In an embodiment, the transformer model architecture comprises a point cloud input module and a text input module. A point cloud encoder module is operatively coupled to the point cloud input module, and a large language model module is operatively coupled to the text input module and the point cloud encoder module and configured to receive data therefrom. A text output module is operatively coupled to the large language model module and configured to output molecular data in line notation format. In some embodiments, the large language model module and the point cloud encoder module may be combined into a single model.

Particularly, the transformer model utilizes a point cloud encoder for concise and order-invariant representation of molecular and protein 3D structures. A textual format is used for generating spatial molecular structures by initially generating chemical-language (e.g., SMILES) representations, followed by atom coordinates in accordance with the chemical language sequence. This format eliminates the reliance on external software for reconstructing chemical bonds and grants the model autonomy in determining the molecular graph. Further, the encoder may incorporate unique features like point embedding calculations and adjustments to position bias computations to suit the characteristics of domain-specific point cloud data.

A novel pre-training method for molecular point clouds is used to distill data from spatial molecular structures datasets, i.e., 3D molecular data. For example, the pre-training approach may include training the model to predict missing parts of incomplete molecular point clouds by using a dropout technique that operates on entire sub-fragments of 3D structures. Strategies used to create input point clouds may include masking specific fragments and/or blurring selected segments. In an embodiment, 3D molecular data comprising a point cloud is fed into the point cloud encoder module in an unsupervised manner. A point description is inferred, by the pretrained point cloud encoder module, based on one or more points in a neighborhood of a point. A 3D position of the point is determined relative to the one or more points in the neighborhood of the point. At least some of the one or more points are masked or blurred. At least some of the masked or blurred point features are predicted by the pretrained point cloud encoder module. Random points are sampled, and distances between the random points are predicted by the pretrained point cloud encoder module. In some embodiments, a mask or blur loss value may be determined based on the prediction of the masked or blurred point features. A distance loss value may be determined based on the prediction of the distances between the random points, and a weighted sum of mask or blur loss and distance loss may be minimized based on the mask loss and distance loss values, respectively.

After fine-tuning the model within both single-task and multi-task frameworks, the model is shown to provide superior performance across several established spatial molecular generation tasks over competing diffusion models regarding the quality of generated samples and efficiency at training time. For example, in experiments conducted on six spatial molecular generation tasks, the transformer model demonstrates superior performance or at least comparable results with an LM baseline and current diffusion approaches.

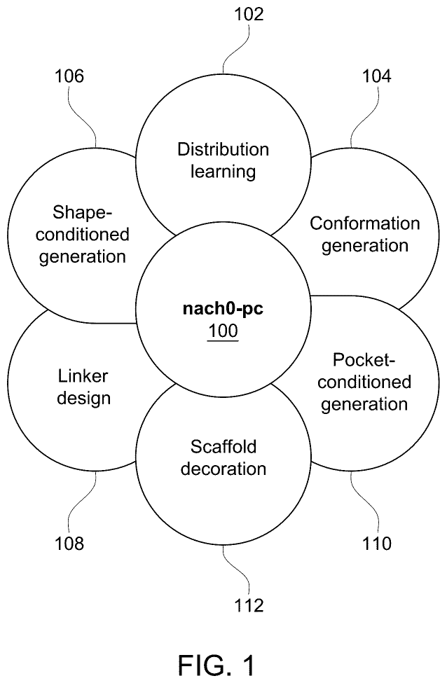

FIG. 1 illustrates an overview diagram of spatial molecular generation tasks in accordance with an embodiment. Each spatial molecular generation task, including conformation generation, pocket-conditioned generation, linker design, may be cast as feeding text and/or point cloud input data into the transformer model 100 and pretraining and training the model 100 to generate a target point cloud represented as text data. Generally, the types of tasks considered herein include text to text tasks (distribution learning 102 and conformation generation 104), molecular point cloud+text to text (shape-conditioned generation 106 and linker design 108), and molecular/protein point cloud+text to text (e.g., pocket-conditioned generation 110 and scaffold decoration 112). Further, one skilled in the art will understand that various other spatial molecular generation tasks also may fall within the scope of the various embodiments.

### Language Models in Chemistry

The sequential nature of molecules enables the use of transformer models and pre-training methods with models like ChemBERTa, T5Chem (T5), ChemFormer, and BARTSmiles, which utilizes masked language modeling for molecular SMILES representations. Recent advancements have introduced domain specific LMs based on T5. MolT5 uses initial pre-training on a collection of molecule SMILES and texts, followed by single-task fine-tuning for molecule captioning (molecule to text) and text-based molecule generation (text to molecule) tasks. On the other hand, Text+Chem T5 is a cross-domain, multi-task T5 model fine-tuned on five tasks, including forward reaction prediction and retro-synthesis. Another recent model, the multi-domain nach0 large language model, undergoes fine-tuning on a diverse set of 28 task-dataset pairs, employing instruction tuning in a multi-task fashion. Unlike MolT5 and Text+Chem T5, nach0 employs separate tokenization for chemical atoms and natural language tokens. BioT5 utilizes custom tokenization for SELFIES and natural language sequences and is fine-tuned on 15 tasks related to molecule and protein property prediction, drug-target interaction, and protein-protein interaction. DrugChat employs a Graph Neural Network (GNN) for encoding molecule graphs, a large LM (LLM), and an adaptor to convert graph representations for LLM compatibility. Following Transformer-M, MolLM offers a unified pre-training framework with a text transformer encoder and a molecular Transformer encoder, pre-trained on molecular graphs, handling both 2D and 3D structures with attention mechanisms incorporating edge features and 3D spatial relationships. One skilled in the art will understand that one or more of these language models may be employed to implement the various embodiments described herein. Further, various other language model may be adaptable to implement the various embodiments and should be considered to fall within the scope of the various embodiments.

### Spatial Molecular Structure Generative Models

The majority of recently published spatial molecular structure generative models are based on the Denoising Diffusion Probabilistic Model (DDPM) paradigm. These models employ a sequential denoising process, wherein the initial step involves the allocation of positions sampled from a Gaussian distribution model, followed by iterative elimination of noise to construct the molecular structure. The Entity Data Model (EDM) follows this methodology to solve spatial molecular distribution learning. However, a drawback of the diffusion approach in generating 3D molecules is its reliance on external software, such as the open source OpenBabel software, to reconstruct molecular bonds based solely on atom coordinates. Even slight errors in generated atom positions can drastically affect the reconstructed molecular graph. This limitation has been addressed in several further works. The MolDiff model integrates an additional bond predictor to guide diffusion and ensure bond consistency alongside 3D atom coordinates. The Motion Diffusion (MDM) model integrates a diffusion model with a SchNet encoder and a scoring network to ensure edge consistency and enhance sample diversity. Diffusion models can be employed in conditional setups by predefining atom types, positions, bonds, and/or freezing some point positions based on 3D conditions. The Geometric Diffusion (GeoDiff) and Torsional Diffusion models use molecular graphs to perform conformation generation tasks. GeoMol is an end-to-end, non-autoregressive, and SE(3)-invariant machine learning approach to generate distributions of low-energy molecular 3D conformers. The DiffLinker and LinkerNet models are 3D equivariant diffusion models which can learn to generate the linker fragment between given disconnected molecular subfragments in a linker design task. These models can find a stable linker and connect the fragments, resulting in a low-energy conformation for the entire molecule. The DiffDec model was proposed for scaffold (molecular core) decoration tasks and generates R-groups for a given molecular scaffold. In a task called shape-conditioned generation, the reference ligand molecule is represented as a shape—a blurred spatial area that approximates the volume inside the molecular surface. The ShapeMol model uses a diffusion approach to suggest new molecules whose shapes closely resemble that of a reference molecule. Pocket-conditioned generation tasks involve creating molecules that fit seamlessly within a designated pocket space, avoiding clashes while establishing interactions to enhance binding affinity. The D3FG and TargetDiff diffusion models can solve this task by designing novel 3D molecules from the scratch that effectively bind to a specified protein pocket. The DecompDiff model proposes to enhance diffusion process with data-dependent decomposed priors, reflecting the natural segmentation of a ligand molecule into functional regions. However, as described above, there is a need for an improved model for handling domain-specific data representing molecular features and spatial data.

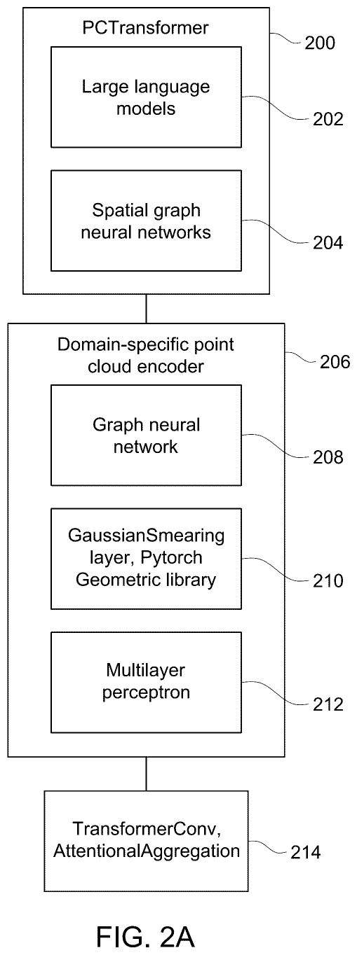

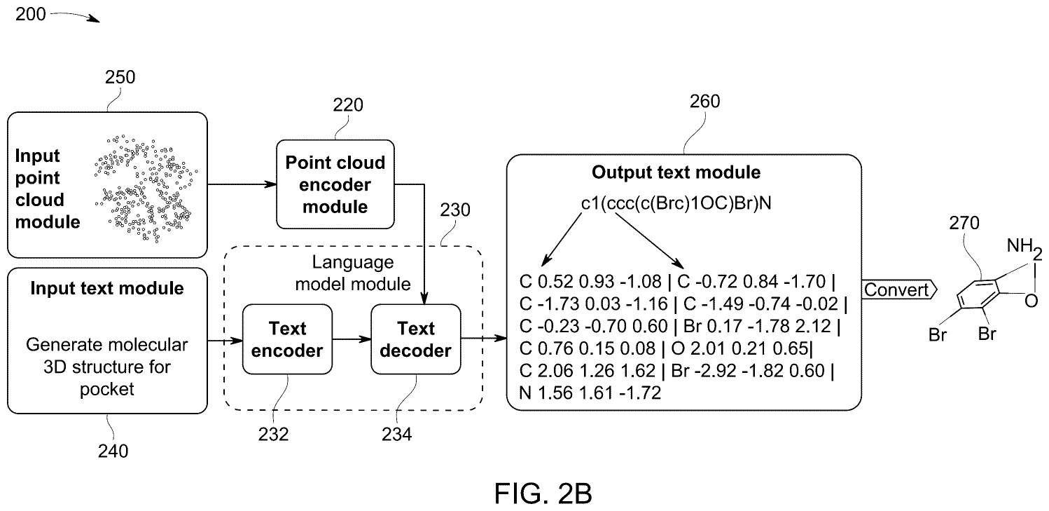

The following sections refer to FIGS. 2A & 2B, which illustrate functional feature-based and modular component-based block diagrams of a point cloud transformer model architecture 200 in accordance with an embodiment.

Referring to FIG. 2A, point cloud transformer model architecture 200 is a multimodal architecture that processes both textual data and 3D molecular point clouds. It receives these two types of input data and processes them to produce the desired output. The textual data undergoes a standard tokenization process, which breaks down the text into smaller pieces, or tokens. This process may be similar to those used in generalized language models such as T5, Bidirectional Encoder Representations from Transformers (BERT), and BART. The 3D molecular point clouds, on the other hand, are processed using a domain-specific point cloud encoder.

Large language models 202, also referred to as pretrained language models or massive language models, are trained on a large amount of unlabeled textual and spatial chemical structures data. These models can understand and interpret complex language patterns and structures, making them ideal for processing the textual data received by the PCTransformer (e.g., sometimes referred to as nach0.pc). The models are pretrained, meaning they have already been trained on a large dataset before being used in the PCTransformer. This pretraining allows the models to process the textual data more efficiently and accurately.

Spatial graph neural networks 204, also known as graph neural networks for spatial data or spatial GNNs, work with the 3D molecular point clouds. These networks are designed to process 3D data, making them ideal for handling the point clouds. The networks work by processing the relative distances between points within the point clouds. This allows the networks to accurately capture the spatial information contained within the point clouds.

Domain-specific point cloud encoder 206 processes the 3D molecular point clouds. This encoder may be specifically designed to handle 3D data, ensuring that the information contained within the point clouds is accurately captured and processed. The encoder maintains invariance to rotations, translations, and reflections in the point embeddings, ensuring that the spatial information within the point clouds is preserved regardless of how the point clouds are oriented or positioned.

Graph neural network 208 processes the relative distances between points within the point clouds. This network is designed to work with relative distances, making it ideal for processing the spatial information contained within the point clouds. The network represents points as nodes in a graph, with features associated with each node. The connectivity of the graph is described using an edges list and edges lengths, which describe the connections between nodes.

GaussianSmearing layer and Pytorch Geometric library 210, also known as generalized layers and generalized libraries, are used to encode the edge lengths of the graph to obtain embeddings. These embeddings are then processed by a multilayer perceptron, also known as an MLP or multilayer neural network 212, to produce the final output of the point cloud encoder: a set of hidden representations, also known as hidden layer values or hidden layer outputs.

TransformerConv and AttentionalAggregation operations 214 are used to update the hidden information of the nodes within the graph. These operations may be performed using the PyTorch Geometric library, which provides a range of tools and functions for working with graph data. The operations aggregate information from connected nodes and edges and read out global information from the whole graph, updating the hidden information of the nodes in the process. The output of these operations is a set of hidden representations, which represent the final output of the point cloud encoder.

Referring to FIG. 2B, a process-based block diagram of point cloud transformer model 200 comprises point cloud encoder module 220, and large language model module 230 comprising text encoder 232 and text decoder 234. In an embodiment, the architecture of transformer model 200 extends the large language model module 230 with a domain-specific molecular point cloud encoder module 220 for generating token embeddings based on 3D molecular data comprising a point cloud. For example, the base text encoder-decoder for large language model module 230 may comprise the T5 architecture described above. For example, the use of such standardized architecture can allow users to train large language model module 230 from scratch or fine-tune a pre-trained large language model module 230 alongside point cloud encoder module 220 for text and point cloud tasks. In one embodiment, large language model module 230 may be initialized with a natural language and chemical language model for multi-domain tasks, e.g., the nach0 LM by Insilico Medicine.

### Input/Output Data Format

In an embodiment, transformer model 200 is configured to receive text data via text input module 240 and, optionally, 3D molecular data via molecular point cloud input module 250 to generate a textual output via text output module 260, which can be converted into a 3D molecular structure 270. In some aspects, the model 200 can consider hydrogen-depleted molecular structures.

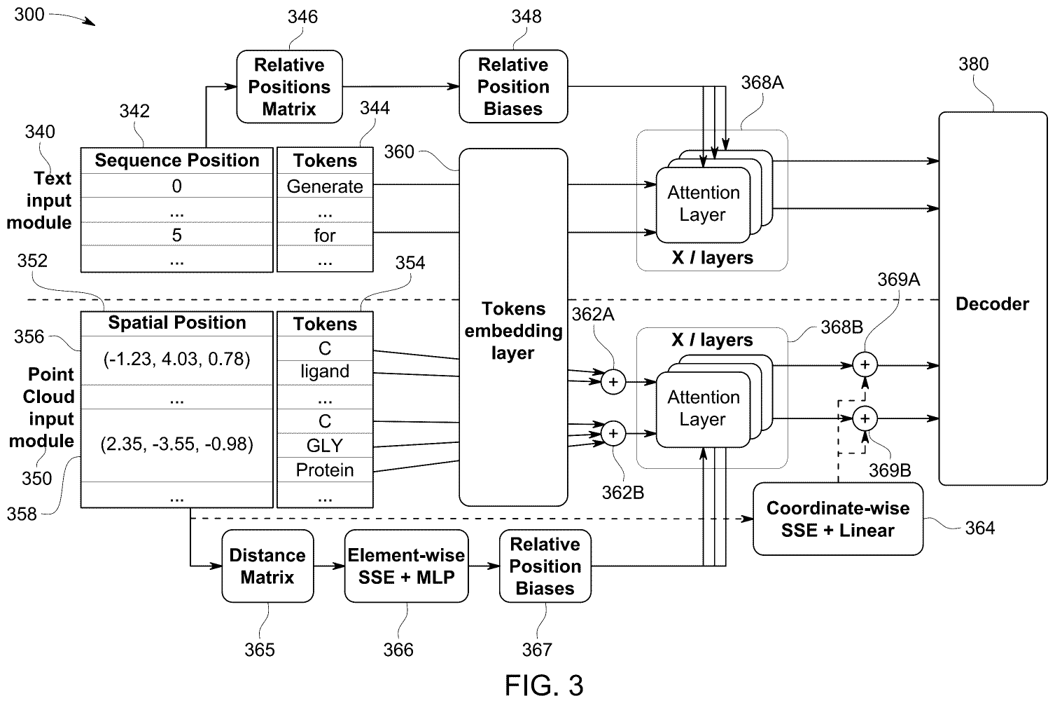

As shown in more detail in FIG. 3, 3D molecular data comprising a point cloud received via point cloud input module 350 may comprise an unordered collection of points, denoted as {pᵢ}, where each point pᵢ=(cᵢ, fᵢ) is described by a spatial position 352, e.g., Cartesian coordinates cᵢ=(xᵢ, yᵢ, zᵢ). Each feature fᵢ^{j} can be seen as a word token or put another way, an unordered set of tokens 354, fᵢ={fᵢ^{j}}, can be used to represent one or more point features. For example, the features 356 of a point corresponding to a ligand's atom may include its atom symbol, atom charge, whether it is in the aromatic ring (e.g., fᵢ={‘atom_N’,‘charge_0’,‘aromatic’, . . . }), etc. For example, a ligand having a positively charged nitrogen atom may be represented as fᵢ={‘ligand’, ‘N’, ‘+’}. In another example, the features for a protein's atom may be extended to include its atom name and its corresponding amino acid name, such as fᵢ={‘pocket’, ‘C’, ‘GLY’, ‘CA’}. Each point feature, e.g., an atom symbol, an atom charge, an atom name, or a corresponding amino acid name may be represented as a word token 354 and can be utilized in textual input and/or output.

3D molecule data may be represented in one or more of a variety of chemical language formats. For example, at least one of the following chemical language formats: a Simplified Molecular-Input Line Entry System (SMILES) format, a Self-referencing Embedded Strings (SELFIES) format, or a XYZ format text, may be used or combined. In one example, 3D molecule data may be represented by combining SMILES and XYZ formats, where the SMILES format may be used to describe the molecular graph, and lines of text may be concatenated in XYZ format, describing each atom's positions in the same order they appear in SMILES. This direct fusion of formats may eliminate the need for external software to reconstruct the molecular graph. In some embodiments, to optimize token count, the number of digits after the decimal point may be restricted (e.g., to two digits) and each coordinate may be tokenized by, for example, splitting the digit at the decimal point (‘−1.23’ to [‘−1’,‘0.23’]), thus each coordinate may be described by two tokens. One skilled in the art will understand than additional formats utilizing additional tokens (e.g., three or more tokens) are possible.

In some embodiments, the 3D molecular data may be down sampled based on one or more prioritized points of the point cloud. For example, points representing a ligand, an alpha carbon (C-alpha) atom, or a terminal atom of a protein amino acid may be retained while other points are omitted. This procedure may reduce the number of points to be processed by the model and may increase model memory efficiency for larger point clouds.

### Point Cloud Encoder

Continuing in reference to FIG. 3, point cloud encoder module 300 may be adapted for text input data received via text input module 340 and, optionally, point cloud input data (e.g., 3D molecular data) via point cloud input module 350. In an embodiment, tokens 354 represent features at specific spatial positions 352. Tokens 354 are embedded via token embedding layer 360, followed by summation pooling 362A and 362B to optimize memory and processing efficiency. In some embodiments, Scalar Sinusoidal Embeddings (SSE) 364 and 366 integrate continuous spatial coordinates (as determined from a distance matrix 365 of spatial positions) and relative pairwise distances, respectively, ensuring translation and rotation invariance.

In an embodiment, point cloud encoder module 300 may comprise a language model text encoder with several changes inspired by the nature of point cloud data. Unlike traditional text, the fundamental data unit is a point rather than a word token. Each point has features and a three-dimensional position.

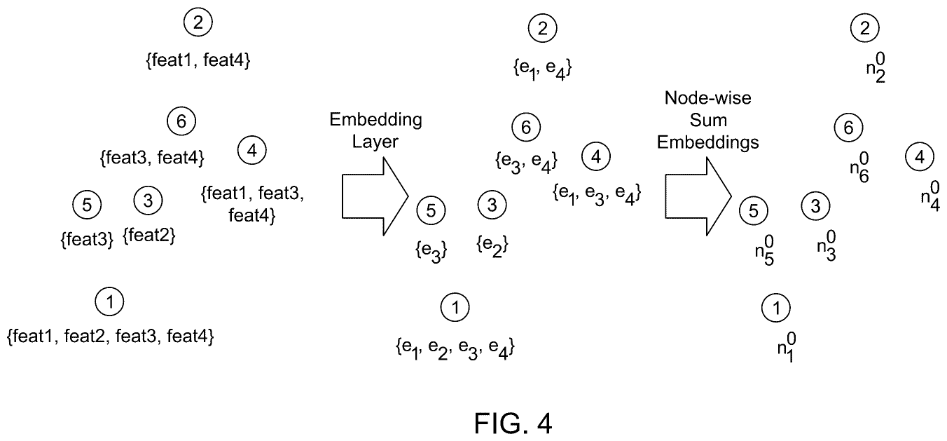

One difference between point cloud encoder 300 and a standard language model text encoder architecture is the point embedding calculation. For natural language, sequentially positioned text 342 is converted to word tokens 344 that are transformed using token embedding layer 360. In an embodiment, token embedding layer 360 also may be applied to 3D molecular data embedded as point tokens. For example, the point cloud features may be converted to hidden vectors by tokens embedding layer 360 and then summed up to form node-wise hidden vectors {nᵢ⁰} with the following formula nᵢ⁰=Σ_{f}_{ᵢ}_{^{j}}_{∈f}_{ᵢ}Embedding(fᵢ^{j}). A process for a point embedding computation is shown in FIG. 4.

Referring back to FIG. 3, following token embedding layer 360, point tokens may be aggregated, e.g., by summation pooling operations 362a and 362b, to consolidate multiple point token embeddings. As a result, a point cloud comprising 3D molecular data may be represented using far fewer hidden vectors than a conventional text representation of the same data. For example, tens of embeddings may be employed to represent a molecular point cloud, while hundreds of embeddings may be used to represent a text token. This modification significantly decreases memory usage while enhancing the speed of attention mechanisms, as shown in graph neural network 368a and 368b, that may be sensitive to input size. For example, attention mechanisms may compute attention biases related to edge features and relative positions between points of a point cloud, and update point embeddings based on the computed attention biases.

In an embodiment, the point spatial coordinates 352 are another difference between point cloud encoder module 300 and a standard language model text encoder architecture. In contrast to a text token 344 where token indices are discrete and form an ascending sequence, each point's spatial position, e.g., spatial positions 356 and 358, in a point cloud 350 may be depicted using continuous Cartesian coordinates. For example, a point cloud relative position biases computation 367 may be modified to embed pairwise distances instead of relative sequence positions, as in a text relative position biases computation 348 performed on a relative text sequence position matrix 346. Further, the spatial coordinates of a point, e.g., point 356 or 358 may be embedded and summed 369a and 369b with point token embeddings before passing embeddings to decoder 380, ensuring that the self-attention layers of graph neural network 368b remain invariant to any point cloud translation or rotation.

As mentioned above, Scalar Sinusoidal Embeddings (SSE) 364 and 366 may be used to embed scalar continuous values of coordinates and distances, respectively. The inclusion of SSEs takes inspiration from positional sinusoidal embeddings and extends the concept to map continuous scalar values into a high-dimensional vector. As represented in the equation below, the input scalar s is divided by wavelengths wᵢ, which are uniformly initialized on a logarithmic grid, and the yielded sine and cosine function values are used to form the resulting embedding vector.

SSE₂ᵢ(s)=sin(s/wᵢ),SSE₂ᵢ₊₁(s)=cos(s/wᵢ)

An example point cloud encoding algorithm in accordance with the various embodiments is shown below:

Point Cloud Encoder Algorithm

```
  Input: Point features fᵢ = {fᵢ^{j}} and coordinates cᵢ = (xᵢ, yᵢ, zᵢ)
  Output: Point embeddings nᵢ^{out}
```

| | |
|---|---|
| 1: nᵢ^{o} = Σ_{f}_{ᵢ}^{j}_{∈f}_{ᵢ} Emb(fᵢ^{j}) | [symbol] embed and aggregate |
| | points features |

2: for l = 1, 2, ... , L do
3: bₕᵢⱼ^{l} = MLPₕ^{l}(SSE(||cᵢ − cⱼ||)) [symbol] for each head h compute relative
attention biases

| | |
|---|---|
| 4: n^{l} = Self AttentionBlock^{l}(n^{l-1}, b^{l}) | [symbol] update point |
| | embeddings |

5: end for
6: nᵢ^{out} = nᵢ^{L} + Linₓ(SSE(xᵢ)) + Lin_{y}(SSE(yᵢ)) + Lin_{z}(SSE(zᵢ)) [symbol] embed
coordinates

### Graph Neural Network

A graph neural network is an alternative way to build architecture of a point cloud encoder. However, any network, technology, or technique may be used for building architecture of a point cloud encoder as descried herein. One constraint on the point cloud encoding procedure is that point embeddings should be invariant to rotations, translations and reflections of point cloud in 3D. To overcome this problem, a graph neural network 368a and 368b that works directly with the relative distances between points rather than raw 3d coordinates is used.

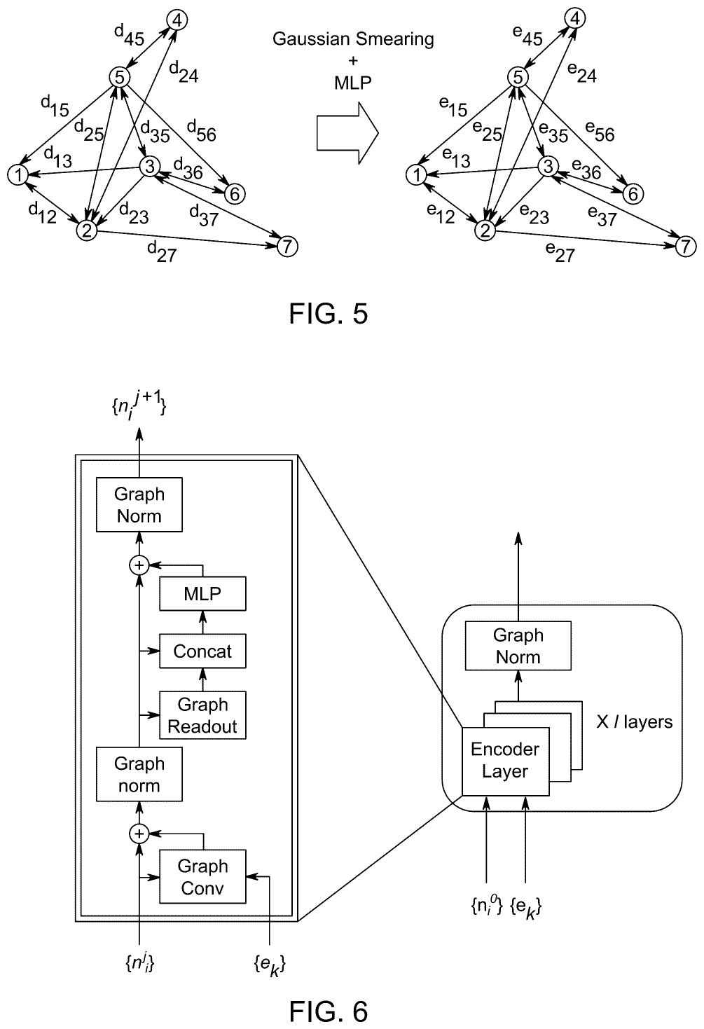

The input of graph neural network 368a and 368b is a graph where the nodes are points (including their corresponding features) and the connectivity of the graph is described by edges list and edges lengths. It can be redundant and resource-consuming to connect all points between each other with edges. Therefore, in practice, connections can be limited to groups of points and the connections between each point and its m nearest neighbors within the point group. If the point belongs to several point groups, a point is connected to the nearest neighbors in each group. So, if point belongs to d point groups, it may be connected to m*d other nodes, however, in practice the nearest neighbors in different groups can overlap and the actual number of connected nodes may be much lower. To get embeddings {eₖ} of edges, edge lengths may be encoded, e.g., with the GaussianSmearing layer from the Pytorch Geometric library, followed by a multilayer perceptron (FIG. 5).

To update the hidden information of nodes, a sequence of l graph neural network layers 368a and 368b may be applied. The structure of a layer can be found in FIG. 6. In an embodiment, each layer (1) aggregates information from connected nodes and edges and (2) processes (reads out) global information from the whole graph. For example, TransformerConv and AttentionalAggregation from the PyTorch Geometric library may be used for these two operations.

The output of point cloud encoder module 300 is the set of hidden representations {nᵢ^{l}} with the size equal to the number of points in the point cloud, one hidden representation vector per point.

### Molecular Point Cloud Pre-Training

In an embodiment, point cloud encoder module 300 may be pretrained in an unsupervised manner on the large set of 3d chemical data to encourage the generalizability of the whole PC transformer model. The pretraining objective for the point cloud encoder is to (1) make an inference of a point description based on points in a neighborhood of the point and (2) understand a relative position of the point in 3D even if the points are not connected by a direct edge.

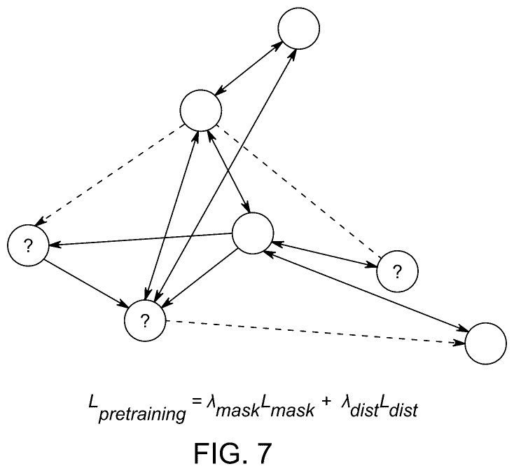

FIG. 7 illustrates a pretraining procedure in accordance with an embodiment. For pretraining, a masking of a fraction of points M can be made with further prediction of the masked point features (as in pretraining techniques used in LLM):

$${L}_{m a s k} = {\sum }_{{f}_{i} ∈ M} {\sum }_{{f}_{i}^{j} ∈ {f}_{i}} CrossEntropyLoss ( M L {P}_{m a s k} ( {n}_{i}^{l} ) , {f}_{i}^{j} )$$

Also, random pairs of points P={(i, j)} are sampled to predict the distances dᵢⱼ=∥cᵢ−cⱼ∥₂ between them. In the practice, the widely used MSE and MAE losses are highly influenced by the pairs of points placed far from each other. To overcome this bias and ignorance of close point pairs, the following loss function is utilized:

$${L}_{dist} = {\sum }_{( i , j ) ϵ P} \vert \frac{{\hat{d}}_{i j}}{{d}_{i j}} - 1 \vert$$

$$Where {\hat{d}}_{i j} = 0.5 * ( M L {P}_{dist} ( {n}_{i} , {n}_{j} ) + M L {P}_{dist} ( {n}_{j} , {n}_{i} )$$

On the pretraining stage the weighted sum of these two losses is minimized:

$${L}_{pretraining} = {λ}_{m a s k} {L}_{m a s k} + {λ}_{dist} {L}_{dist}$$

In an embodiment, the pretrained language model module and point cloud encoder modules can be fused together in one model. To form a fused text-point cloud input of the model, point cloud {pᵢ} is encoded by point cloud input module 350 to get point embeddings {nᵢ^{l}}. Simultaneously, embeddings {wₖ} of input text sequence {tₖ} may be prepared by text input module 340. Notably, both {nᵢ^{l}} and {wₖ} may be unordered sets of embeddings since a point cloud does not have an ordered set of points and the text embeddings incorporate a sequence position by adding positional encodings to each token embedding. To form the text-point cloud fused input, these two sets can form a union: {vₘ}={nᵢ^{l}}∪{wₖ}. The fused input may then be passed to the large language model module (e.g., LLM module 230). For example, the language model may comprise an encoder-decoder transformer model.

Depending on the generation task, the molecular point cloud may require additional preprocessing steps like downsampling, augmenting or adding noise. In the linker design task, parts of a molecule are removed, and models are trained to recover the removed parts. For example, the BRICS algorithm may be used to split a molecule into several fragments and remove a random number of fragments (at least one should remain). For shape-conditioned generation, random fragments may be blurred rather than fully removed. In this task, chosen atoms are duplicated several times and noise with high variance is added.

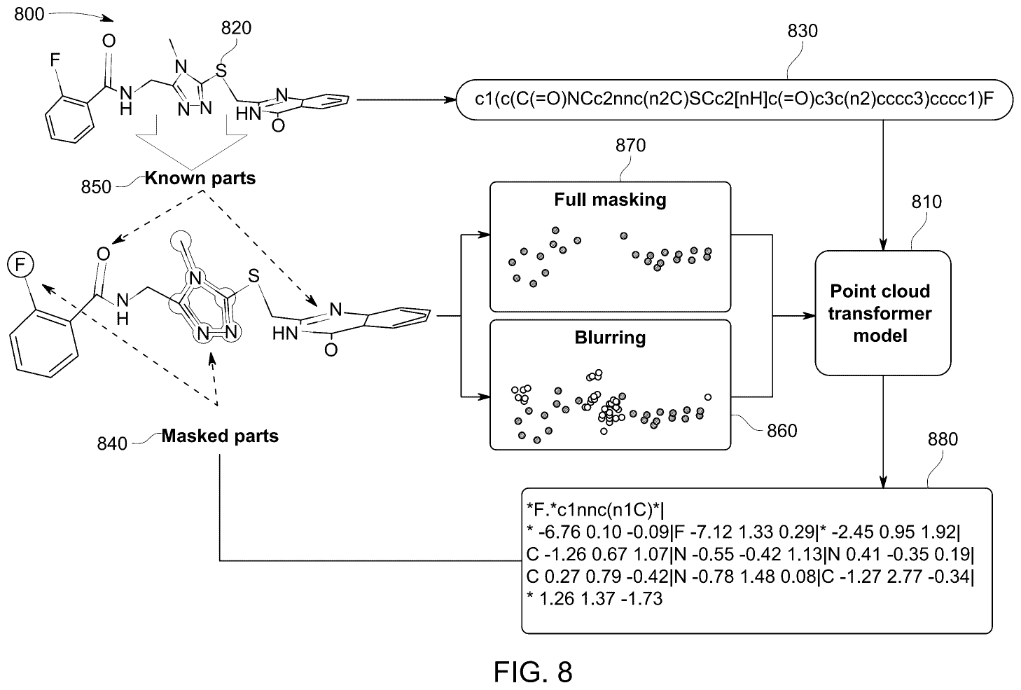

FIG. 8 illustrates a block diagram of a pretraining process 800 for a point cloud transformer model in accordance with an embodiment. For example, the point cloud encoder module 810 may be pretrained using spatial molecular structures 820 and SMILES representations 830 thereof.

In an embodiment, input spatial molecular structure datasets may be formed by (i) removing selected fragments and (ii) obfuscating chosen segments by omitting the features of a point (such as atomic symbol, charge, and valency), replicating the point multiple times, and introducing Gaussian noise to the coordinates of these copies. For example, to reconstruct randomly missing molecular fragments 840 of a molecular structure having known parts 850, a blurring operation (e.g., by Gaussian noise) 860 may be used to obscure portions of corresponding 3D point clouds, and a masking operation 870 may be used to omit point features of corresponding 3D point clouds. Point cloud encoder module 810 may be configured to distill knowledge from unlabeled spatial molecular structure datasets comprising a point cloud. In an embodiment, point cloud encoder module 810 may be feed with incomplete spatial molecular structure datasets and trained to reconstruct the missing pieces. Particularly, a dropout training method may operate with whole subfragments of molecules rather than individual atoms. For example, the BRICS algorithm may be used to split a molecular representation into several fragments and randomly select a subset of these fragments with some predefined probability (ensuring that at least one is chosen) and exclude them from the molecular representation. Further, the pre-training process may be flexible, handling both datasets comprising spatial molecular structures and datasets with protein pockets and ligand pairs. In the latter, for example, the ligand may be masked while the protein pocket remains unchanged in the input point cloud.

As shown in block 880, model 810 may be configured to generate the missing molecular fragments 840 during pre-training, including their SMILES representations, attachment points, and atom coordinates. During pre-training, the model's task is to accurately recover the absent and/or blurred components, specifying their SMILES representations as well as their atomic coordinates. For example, each recovered fragment may include a connecting point indicated by the symbol ‘*’. In cases where the model should reconstruct multiple missing fragments, the fragments are separated with the ‘.’ token. In experiments described below, the pretraining process 800 enhances the model's performance on downstream tasks. An analysis of the impact of pretraining process 800 is included below in Example H).

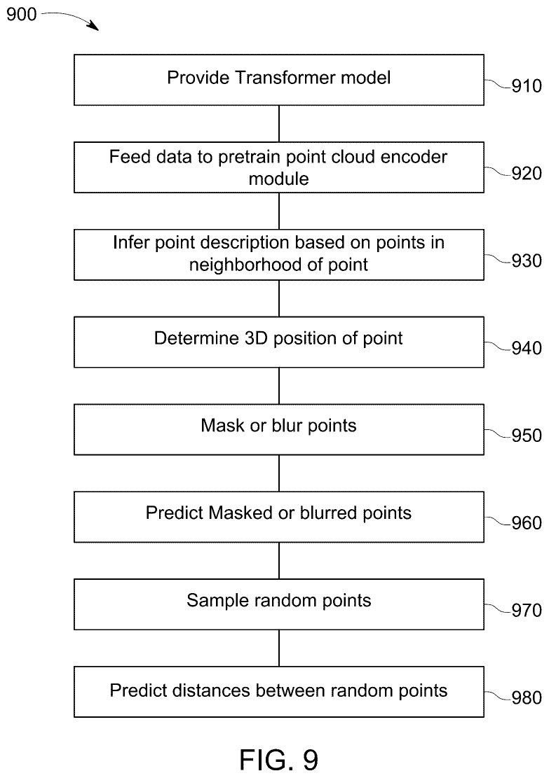

FIG. 9 illustrates a flowchart 900 for pretraining a transformer model in accordance with an embodiment. At block 910, a transformer model is provided. For example, the transformer model, e.g., transformer model 200 described above, may comprise a point cloud input module, a text input module, a point cloud encoder module operatively coupled with the point cloud input module, a large language model module operatively coupled with, and configured to receive data from, the text input module and point cloud encoder module, and a text output module configured to output molecular data in line notation format. In some embodiments, the large language model module and the point cloud encoder module may be combined as a single module, e.g., as illustrated by point cloud encoder module 300.

At block 920, the point cloud encoder module is pretrained in an unsupervised manner using 3D molecular data comprising a point cloud. For example, the point cloud encoder module 810 may be pretrained using obtained representations of spatial molecular structures 820 and SMILES representations 830 thereof. At block 930, a point description is inferred, by the point cloud encoder module, based on one or more points in a neighborhood of the point. A 3D position of the point is determined relative to the one or more points in the neighborhood of the point at block 940. At block 950, at least some of the one or more points are masked or blurred, and at least some of the masked or blurred point features are predicted by the point cloud encoder module at block 960. In some embodiments, various points may be masked and blurred, as shown in FIG. 8. For example, a pretraining data point cloud may be encoded into one or more point embeddings, and embeddings of an input text sequence may be prepared (see FIG. 3). The one or more point embeddings and the input text sequence embeddings may be combined to obtain a fused input, and the fused input may be feed into the point cloud encoder module.

At block 970, random points are sampled, and distances between the random points are predicted by the point cloud encoder module at block 980. In some embodiments, a mask or blur loss value may be determined based on the prediction of the masked or blurred point features. For example, a distance loss value may be determined based on the prediction of the distances between the random points, and a weighted sum of mask or blur loss and distance loss may be minimized based on the mask loss and distance loss values, respectively.

### Experiments

The proposed PC transformer model is evaluated on several molecular generation tasks. MOSES, GEOM and CrossDocked2020 molecular datasets have been used to train and validate model on distribution learning, linker design and shape-conditioned generation tasks.

MOSES is a benchmarking platform that provides a molecular dataset and a set of metrics to compare molecular generative models trained on this dataset. The dataset contains around two million of molecules divided into train, test, and scaffold test parts. Each molecule is represented in text SMILES format. This unlabeled dataset is used to pretrain the model in an unsupervised way so it can understand the distribution of synthetically accessible and druglike molecules along with SMILES format structure and rules.

GEOM datasets provide molecular conformation ensembles computed with CREST and GFN2-xTB software combination. The GEOM dataset contains two subsets: (1) computed conformations for 133,258 molecules from QM9 dataset, and (2) computed conformations for 304,466 molecules from AICures challenge. All conformations were computed for a vacuum environment.

CrossDocked2020 dataset is an extensive library of molecular poses docked in the variety of protein pockets. It contains 22.5 million poses of ligands docked into multiple similar binding pockets across the Protein Data Bank.

Experiments were preformed to assess the ability of the PC transformer model to solve a variety of molecular generation tasks. Substructures from the MOSES dataset were employed to pretrain the text part of the proposed model. During the pretraining stage, the model is trained on the rules of SMILES format and the distribution of drug-like molecule.

### Shape-Conditioned Generation

For shape-conditioned generation, the model is trained to recover a molecule from a blurred region of 3D space (called a “shape”) where the molecule is located. In practice, some parts of the molecular point cloud are blurred, e.g., with intense noise, so it is difficult even for human experts to recognize the blurred fragments. Models trained for this task can be used for generating known ligand alternatives that fit spatially but have a different molecular structure. If desired, some parts of the molecular point cloud can be preserved by keeping them unaltered and non-blurred.

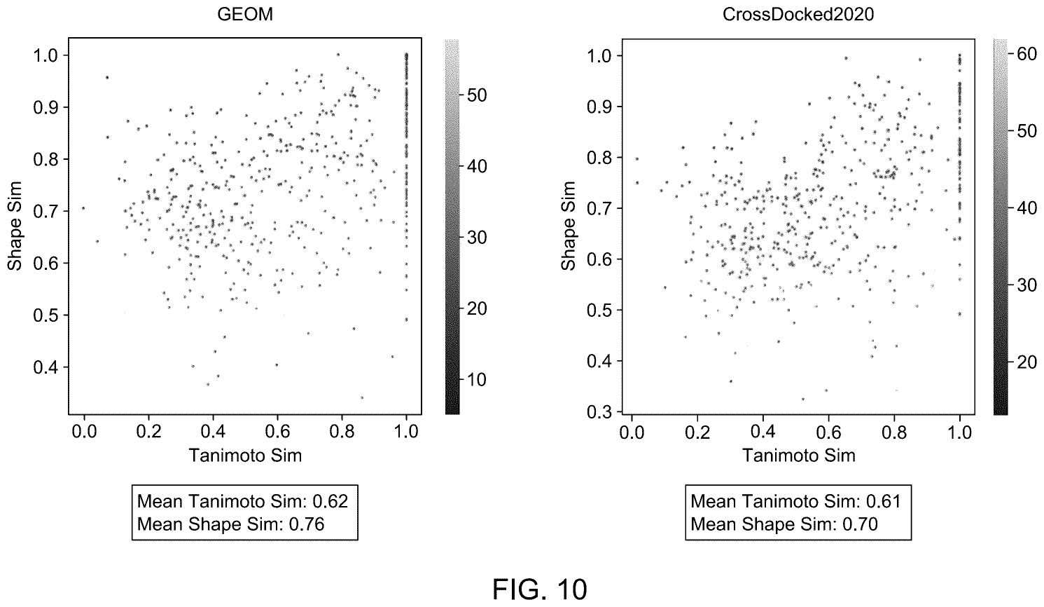

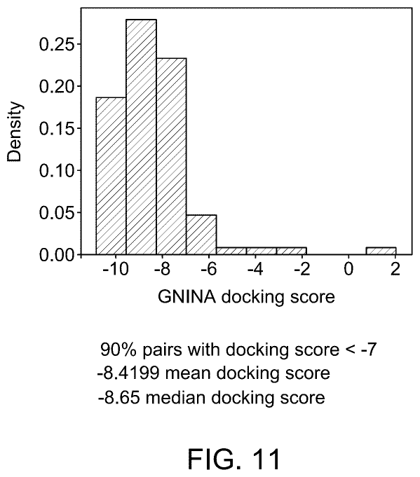

Training and validations of the model were conducted on GEOM and CrossDocked2020 datasets. The results (FIG. 10) showed that model produces alternatives with 0.62(GEOM)/0.61(CrossDocked2020) mean Tanimoto similarity and 0.76(GEOM)/0.70(CrossDocked2020) mean shape similarity. The model preserves non-blurred fragments in 97.32% (GEOM)/92.91% (CrossDocked2020) cases (FIG. 11).

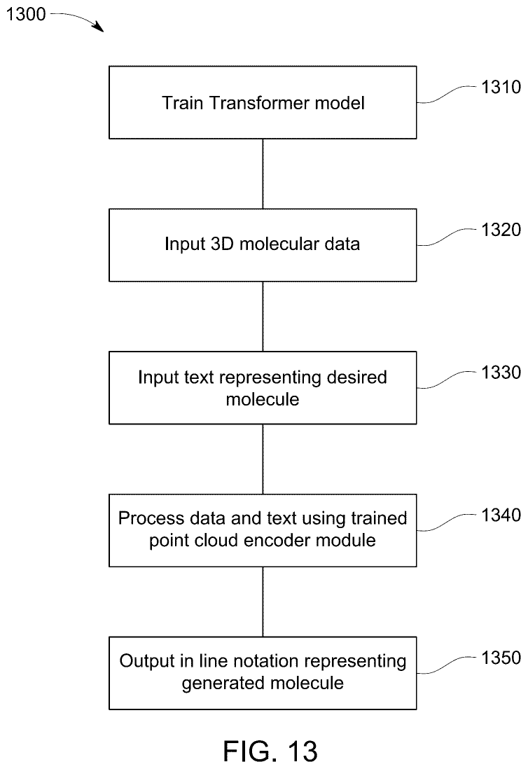

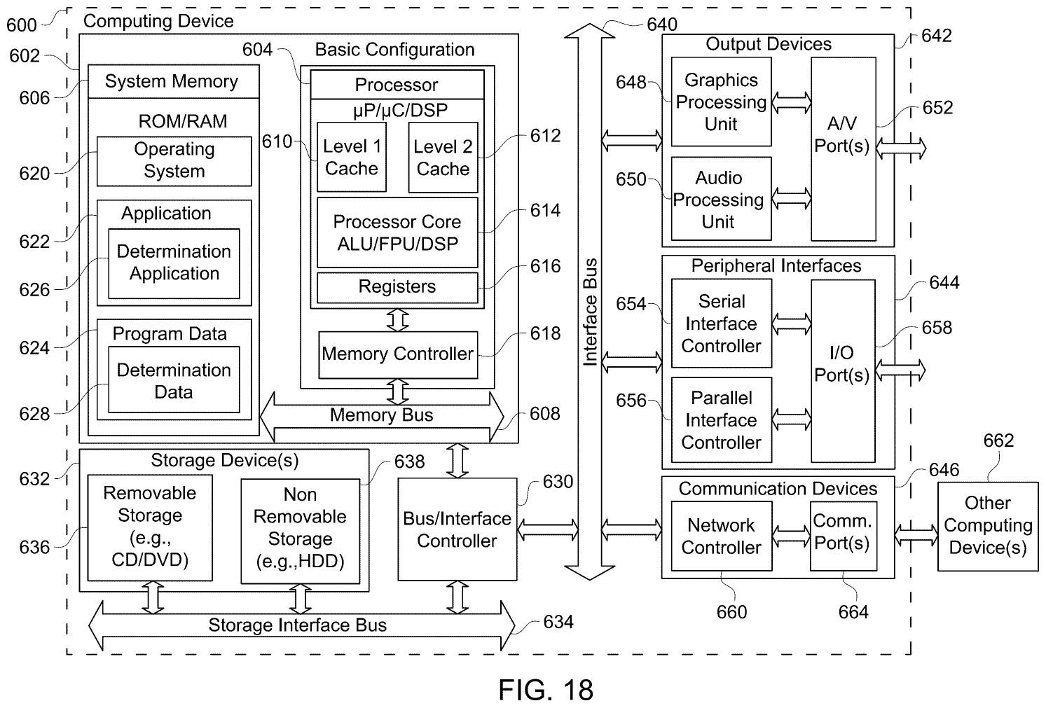

FIG. 13 illustrates a flowchart of shape-conditioned generation utilizing a trained transformer model in accordance with an embodiment. For example, a computer system as described in FIG. 18 below, may comprise a trained transformer model and one or more non-transitory computer readable media storing instructions that, in response to being executed by one or more processors, can cause the computer system to perform one or more of the operations described in FIG. 13, including, for example, operating the transformer model or pretraining thereof or implementation thereof for shape-conditioned generation of at least one molecule.

Referring to FIG. 13, shape-conditioned generation 1300 comprises, at block 1310, training a transformer model to recover a molecule of a molecular point cloud from a blurred region of 3D space where the molecule is located. For example, some parts of a molecular point cloud may be blurred and a selected portion of the molecule may be unaltered and not blurred. At block 1320, 3D molecular data comprising a point cloud is inputted via a point cloud input module. At block 1330, text representing a desired molecule is inputted via a text input module. The 3D molecular data and text are processed using the trained point cloud encoder module at block 1340, and a text representing a molecule is outputted in line notation at block 1350.

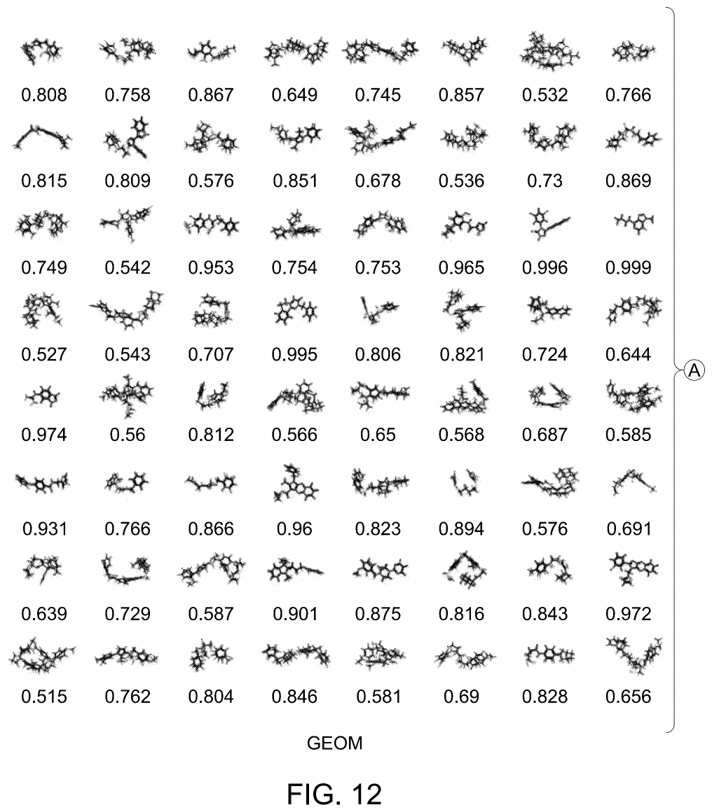

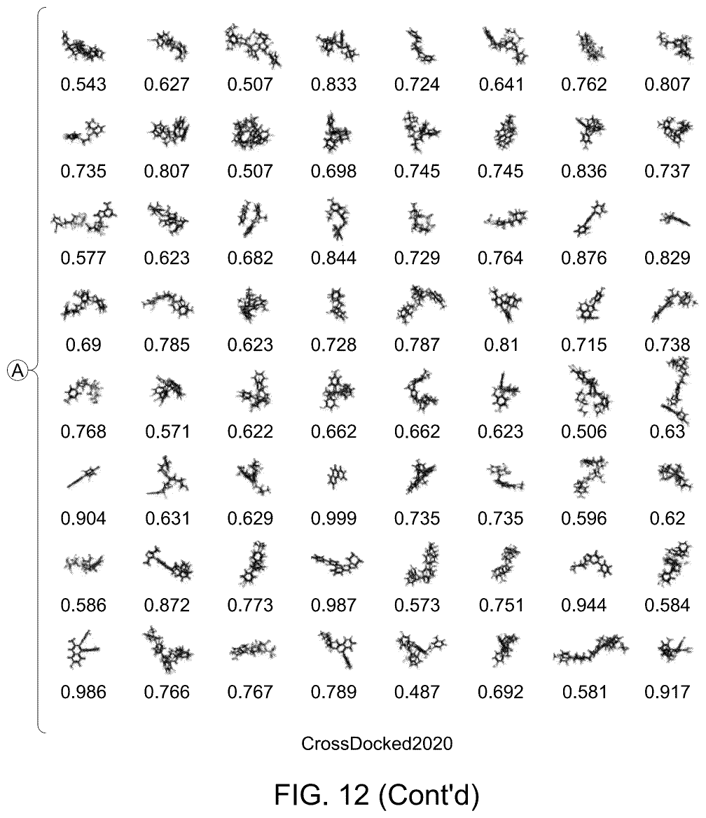

Examples of shape-conditioned molecules generated as described herein can be found on FIG. 12.

### Linker Design

In the linker design task, the model is trained to recover the removed part of the molecule. This task is more complex than shape-conditioned generation since the model does not receive a spatial description (size, curvature, etc.) of removed fragments. Similar to the previous task, a model trained to solve linker design problems is able to generate molecular alternatives for a known ligand. It can be also applied in cases where no known ligand is known but there is knowledge of where active fragments should be located in the 3D space.

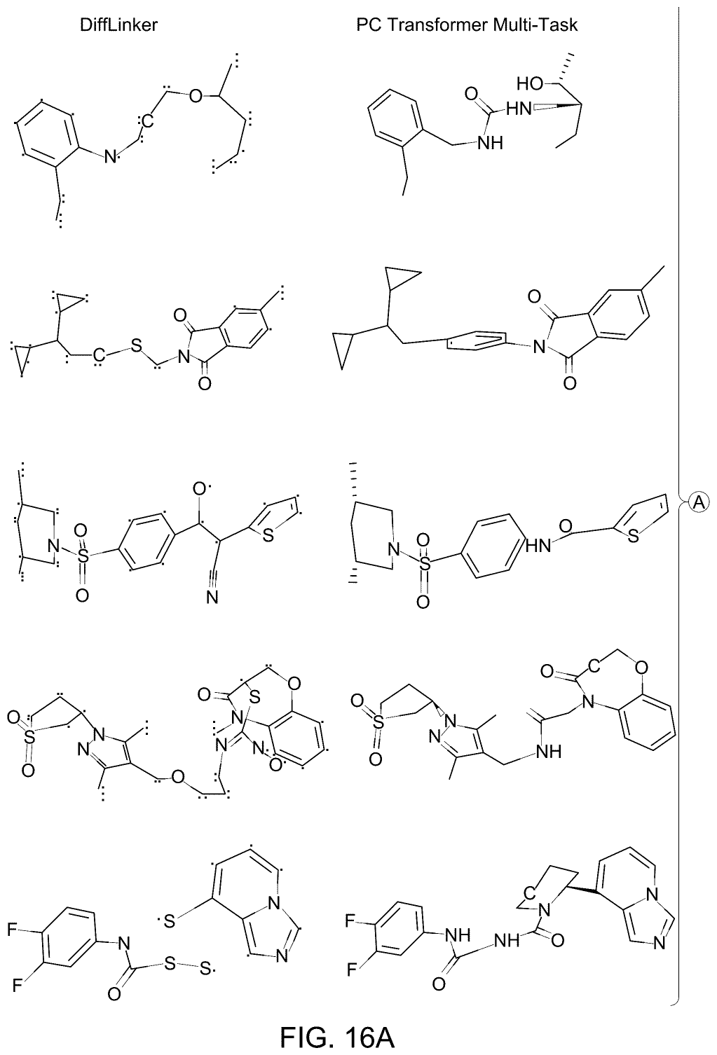

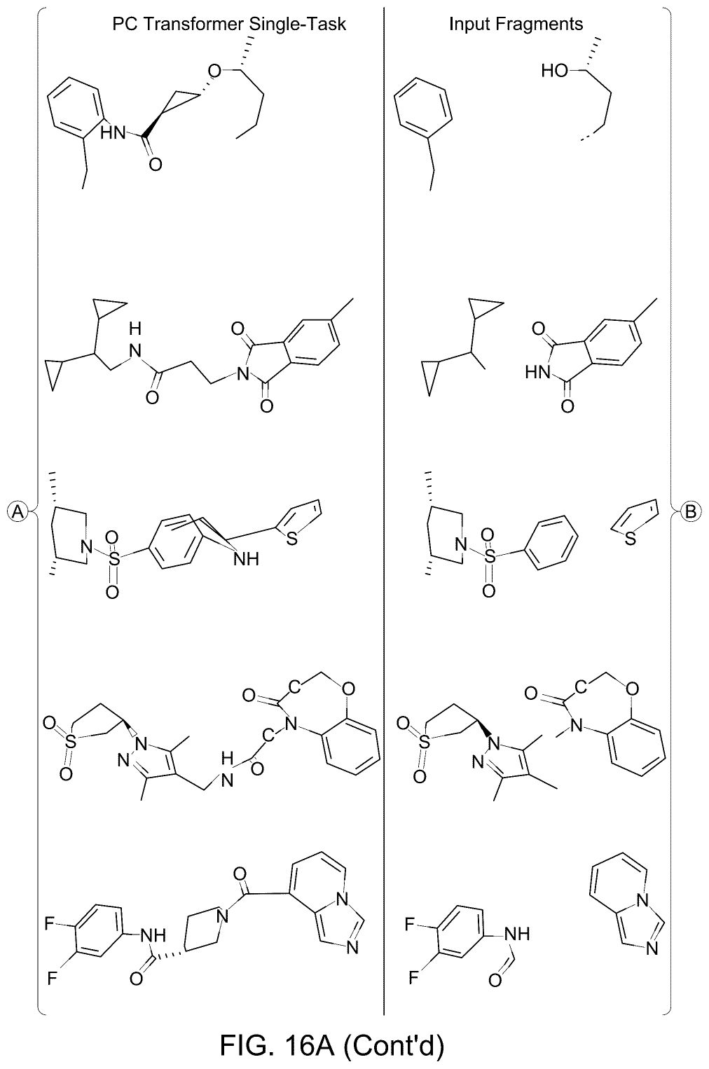

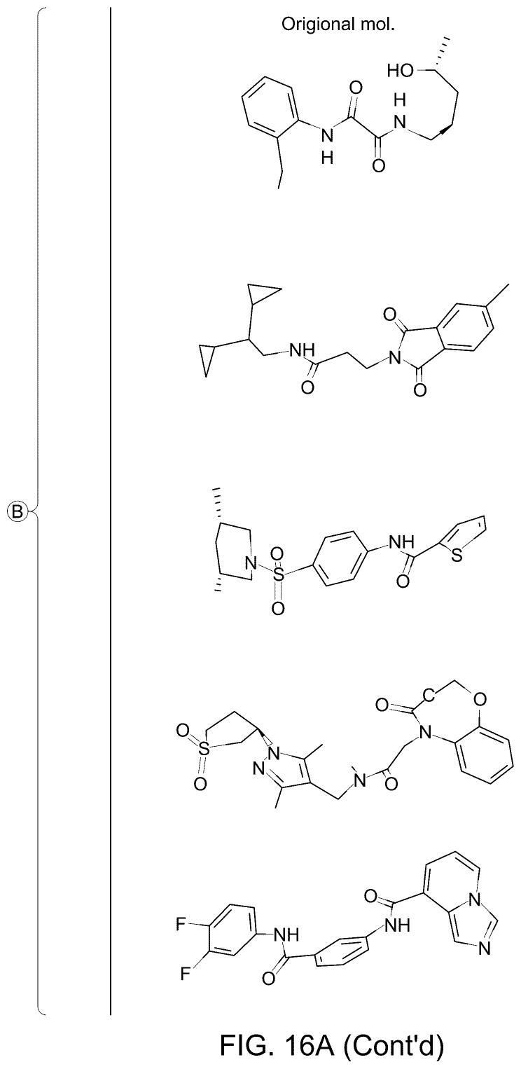

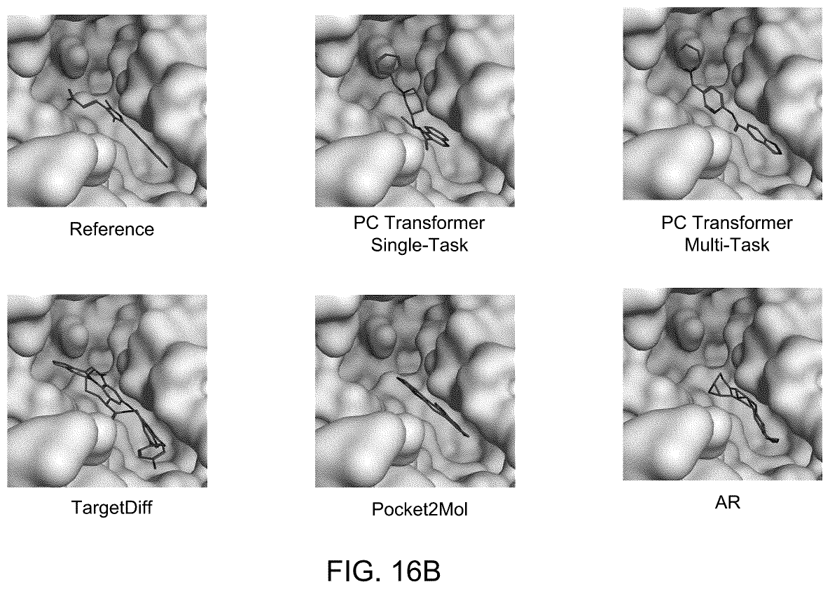

Training and validations of the proposed model were conducted on the GEOM dataset for solving linker design problem. In 96.39% cases the generated molecules contain desired fragment conditions. Examples of aligned ground-truth/generated pairs and fragment conditions/generated molecules can be found on FIG. 16A.

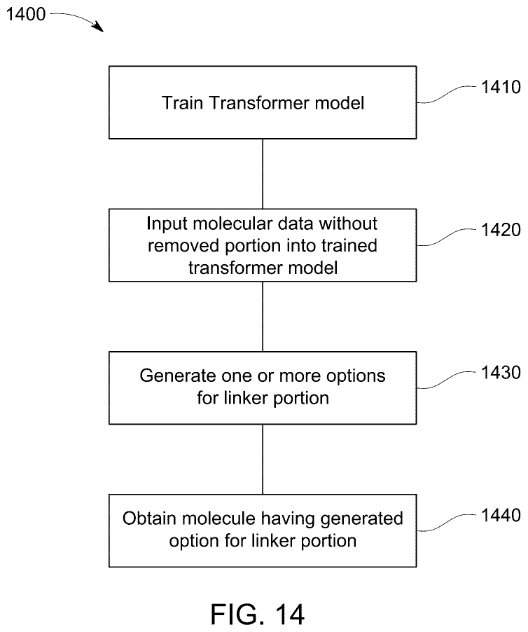

FIG. 14 illustrates a flowchart of linker design generation utilizing a trained transformer model in accordance with an embodiment. For example, a computer system as described in FIG. 18 below, may comprise a trained transformer model and one or more non-transitory computer readable media storing instructions that, in response to being executed by one or more processors, can cause the computer system to perform one or more of the operations described in FIG. 14, including, for example, operating the transformer model or pretraining thereof or implementation thereof for linker design generation of at least one molecule.

Referring to FIG. 14, linker design generation 1400 comprises training a transformer model to recover a removed part of a molecule when the transformer model architecture does not receive a spatial description of the removed part of the molecule at block 1410. At block 1420, data representing one or more molecules is inputted into the trained transformer model. At block 1430, the trained transformer model is used to generate one or more options for a linker portion of a molecule to replace the removed part of the molecule. At block 1440, one or more molecules having the generated one or more options are obtained for the linker portion of the molecule.

The foregoing summary is illustrative only and is not intended to be in any way limiting. In addition to the illustrative aspects, embodiments, and features described above, further aspects, embodiments, and features will become apparent by reference to the drawings and the following detailed description.

### Experimental Results

The effectiveness of the point cloud transformer model has been evaluated across the following spatial molecular generation tasks: (i) 3D molecular structures generation (spatial molecular distribution learning, conformation generation); (ii) molecular point cloud completion (linker design, scaffold decoration); (iii) shape-conditioned generation; and (iv) pocket-conditioned generation. See Example sections B, C, D, and F below for details on datasets, models, and metrics used to obtain the experimental results.

### Spatial Molecular Distribution Learning and Conformation Generation Tasks

3D molecular structures generation evaluates the model's ability to generate structurally diverse and physically plausible spatial molecular objects.

The spatial molecular distribution learning task assesses whether the model can produce novel 3D molecular structures whose distribution is close to a ground truth. A high-quality dataset, e.g., the GEOM-Drugs (also referred to herein as “GEOM”) dataset, was used to offer conformation ensembles generated using metadynamics in CREST. The following valid molecules were evaluated from five perspectives: drug likeness, 3D substructure, bond, ring distributions, and the Root Mean Square Distribution (RMSD) to the template conformation.

In the conformation generation, the focus is on generating plausible conformations for a given molecular graph. The conformations of a molecule are its energetically favorable 3D structures, each representing a local minimum on the potential energy surface. Like the previous task, generated conformation ensembles were evaluated on the GEOM dataset. The Average Minimum RMSD (AMR) and Coverage metrics were followed and employed. These metrics evaluate Recall (R), indicating the extent to which the generated ensemble covers the ground-truth ensemble, and Precision (P), measuring the quality within the generated conformations.

Consistent with the mentioned baselines, 2K conformations were generated for a molecule with K ground truth conformations and train/validation/test splits were utilized. Spatial molecular distribution learning task baselines were retrained on this data split and the metrics on 10K sampled objects were reported for fair comparison. That included the retrained MolDiff model using all necessary elements, whereas it originally supported molecules with only major ones (C, N, O, F, P, S, and Cl).

The generative capabilities of the model, trained in both single-task and multi-task settings, were assessed in comparison with (i) the MolDiff model for the first task and (ii) the GeoMol, GeoDiff, and Torsional Diffusion (Tor. Diff.) models for the second task. An LM baseline model was adopted for both tasks based on the open source LLaMa model architecture from HuggingFace. The Open LLaMa implementation was utilized and training was conducted on the same GEOM-Drugs dataset (See configuration details in Example E.2).

The results for two tasks are presented in Tables 1 and 2.

TABLE 1
Spatial molecular distribution learning performance metrics on GEOM-DRUGS.

| | | State-of-the-art | | PC Transformer | |
|---|---|---|---|---|---|
| | | MolDiff | | Multi- | Single- |
| Group | Metrics | [35] | OpenLLaMA | Task | Task |
| Druglikeness | QED(↑) | 0.679 | 0.655 | 0.771 | 0.664 |
| | SA(↑) | 0.875 | 0.831 | 0.872 | 0.848 |
| | Lipinski (↑) | 4.981 | 4.96 | 4.992 | 4.938 |
| 3D | JS. bond lengths (↓) | 0.442 | 0.185 | 0.204 | 0.146 |
| substructures | JS. bond angles (↓) | 0.172 | 0.125 | 0.108 | 0.101 |
| | JS. dihedral angles (↓) | 0.198 | 0.136 | 0.132 | 0.113 |
| Bonds | JS. # bonds per atoms (↓) | 0.121 | 0.031 | 0.233 | 0.094 |
| | JS. freq. bond types (↓) | 0.170 | 0.028 | 0.052 | 0.041 |
| | JS. freq. bond pairs (↓) | 0.153 | 0.037 | 0.040 | 0.033 |
| | JS. freq. bond triplets (↓) | 0.137 | 0.047 | 0.054 | 0.041 |
| Rings | JS. # rings (↓) | 0.079 | 0.028 | 0.270 | 0.036 |
| | JS. # n-sizedrings (↓) | 0.102 | 0.010 | 0.058 | 0.024 |
| | # Intersecting rings (↑) | 8 | 9 | 9 | 8 |
| RMSD | Mean RMSD (↓) | 0.993 | 1.231 | 1.087 | 1.347 |

TABLE 2
Generated conformer ensembles quality on GEOM-DRUGS

| | Recall | | | | Precision | | | |
|---|---|---|---|---|---|---|---|---|
| | COV (↑) | | MAT (↓) | | COV (↑) | | MAT (↓) | |
| Method | Mean | Med | Mean | Med | Mean | Med | Mean | Med |

State-of-the-art

| | | | | | | | | |
|---|---|---|---|---|---|---|---|---|
| RDKit | 38.4 | 28.6 | 1.058 | 1.002 | 40.9 | 30.8 | 0.995 | 0.895 |
| ETKDG [54] | | | | | | | | |
| GeoMol [40] | 44.6 | 41.4 | 0.875 | 0.834 | 43.0 | 36.4 | 0.928 | 0.841 |
| GeoDiff [38] | 42.1 | 37.8 | 0.835 | 0.809 | 24.9 | 14.5 | 1.136 | 1.090 |
| Tor. Diff. | 72.7 | 80.0 | 0.582 | 0.565 | 55.2 | 56.9 | 0.778 | 0.729 |
| [39] | | | | | | | | |
| OpenLLaMa | 37.1 | 29.2 | 0.909 | 0.897 | 30.3 | 20.0 | 1.114 | 1.071 |
| [53] | | | | | | | | |

PC Transformer

| | | | | | | | | |
|---|---|---|---|---|---|---|---|---|
| Multi-Task | 50.1 | 50.0 | 0.761 | 0.753 | 27.7 | 16.7 | 1.110 | 1.114 |
| Single-Task | 57.7 | 59.5 | 0.705 | 0.694 | 32.4 | 23.1 | 1.031 | 1.034 |

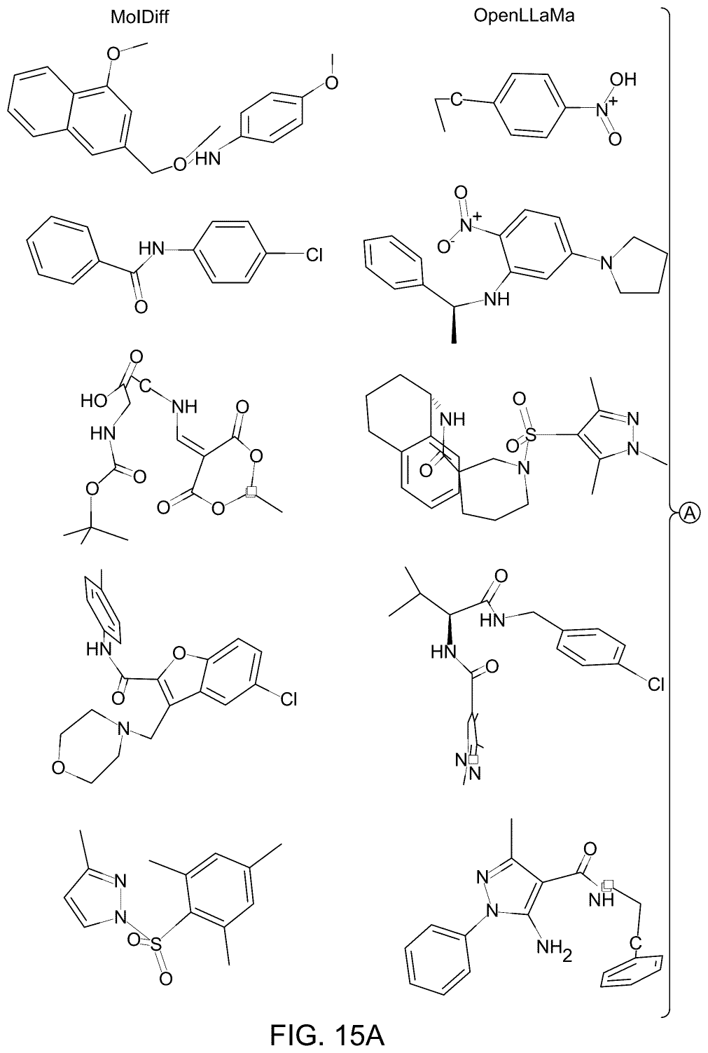

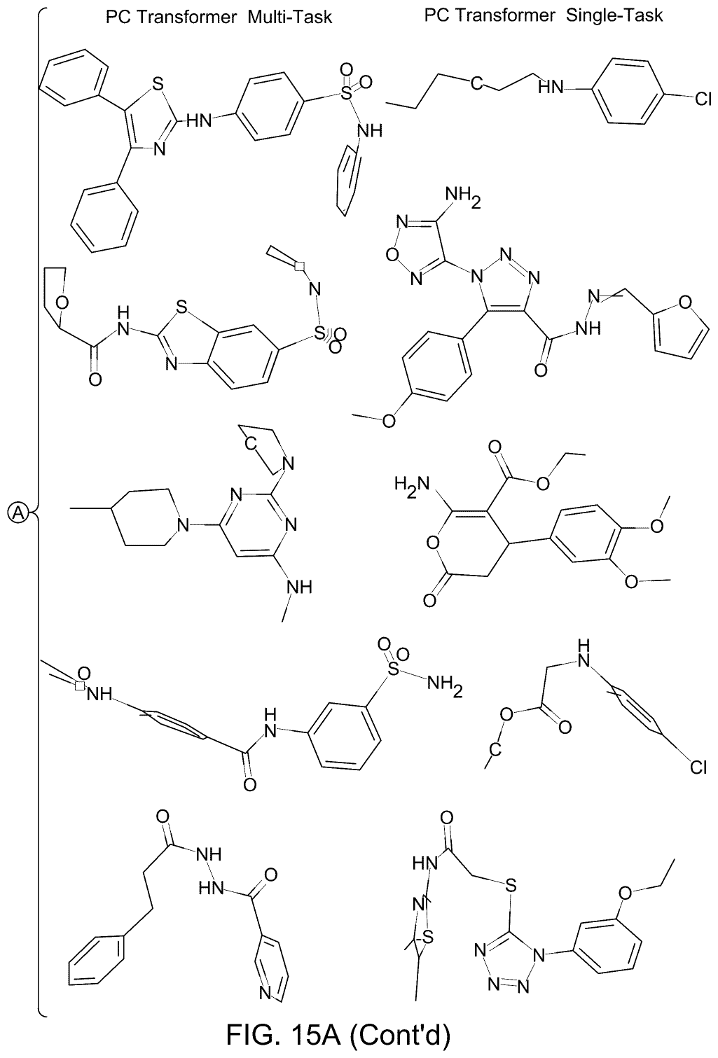

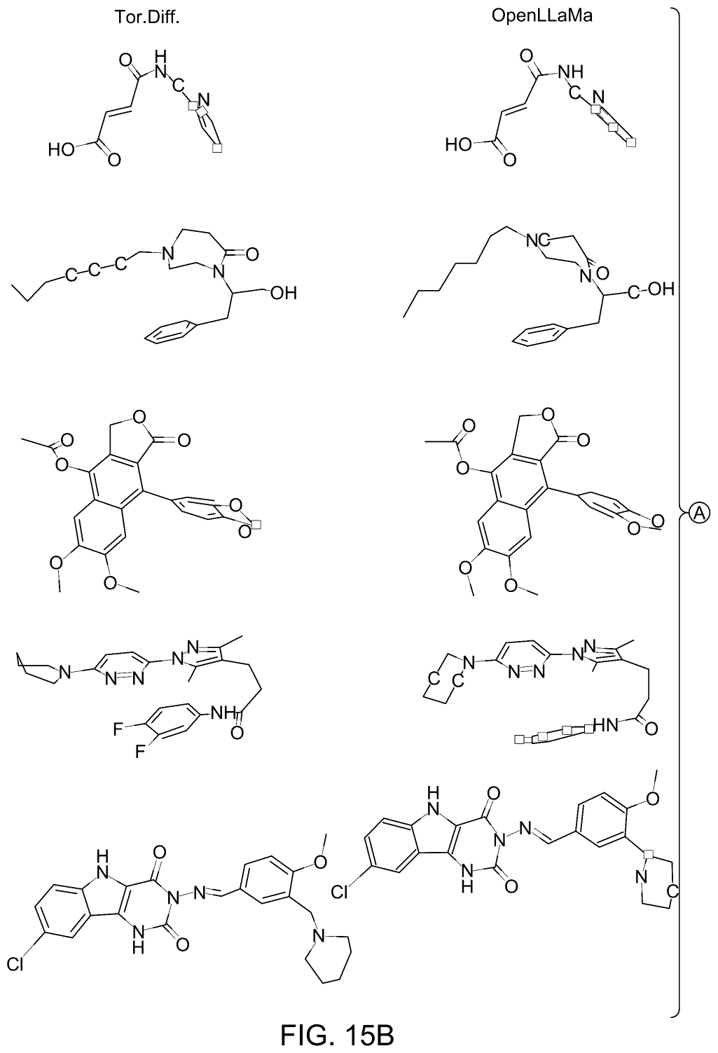

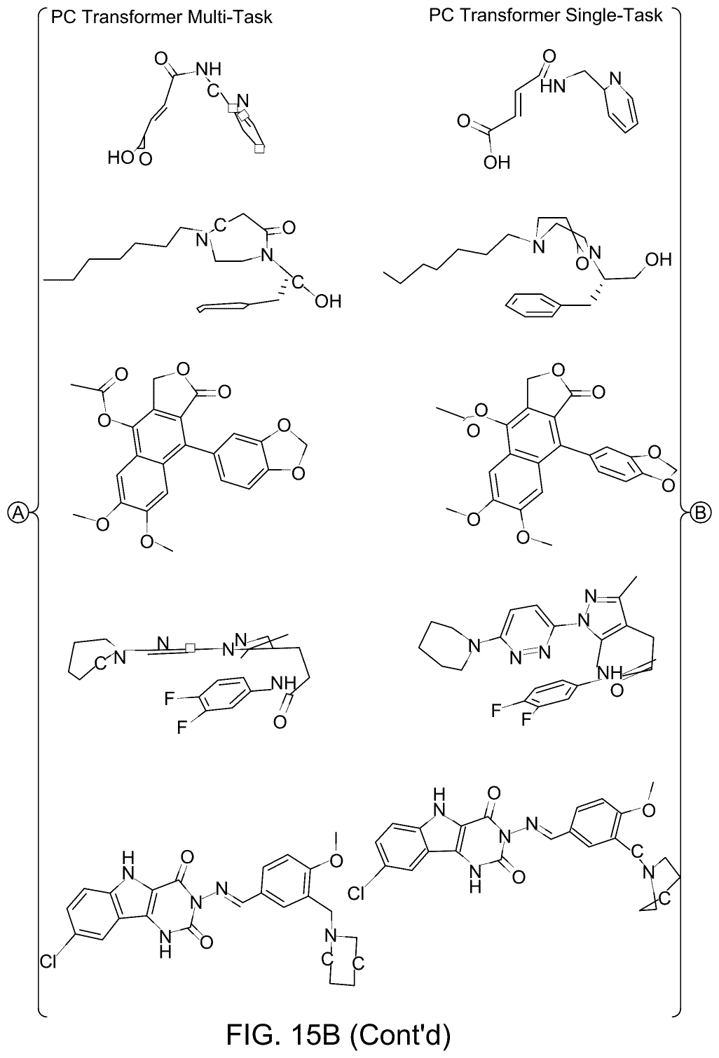

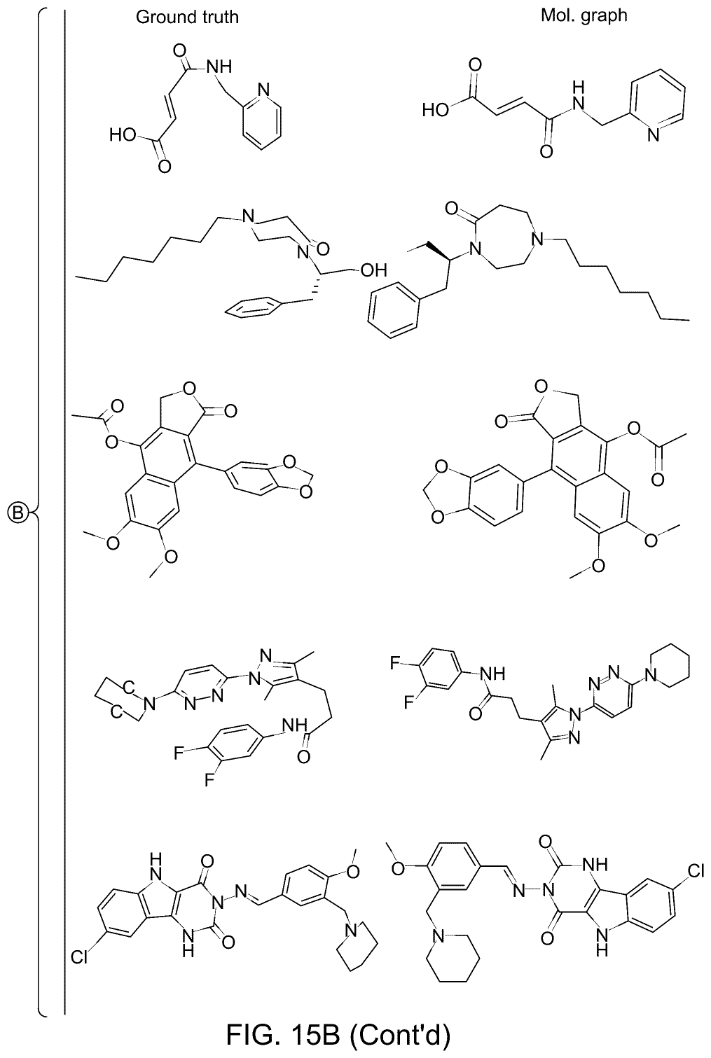

As shown in Table 1, the LMs (PC transformer model and OpenLLaMa) outperform the MolDiff diffusion model, producing a molecular distribution closer to the ground-truth in terms of 3D substructure, bond, and ring distributions. Particularly, the PC transformer model produces more accurate molecular structures, outperforming OpenLLaMa on the 3D substructures group while being better or close on other metrics (FIGS. 15A & B). On the conformation generation task, the only model outperforming the PC transformer model on the whole set of metrics is the Torsional Diffusion model, which utilizes samples from external software (RDKit) as an initial generation point on the inference stage. Nevertheless, the PC transformer model shows stronger results than all other purely neural network baselines that produce molecular conformation from scratch.

### Linker Generation and Scaffold Decoration Tasks

Molecular point cloud completion (linker design, scaffold decoration) tasks assess the ability of models to complete disjoint or partially defined molecular structures.

In linker design tasks, models operate with several disconnected fragments and should produce small molecular structures that spatially and chemically connect to the given fragments (as shown in FIG. 16A) and complete into one connected chemical structure. A subset of the ZINC dataset, comprising 250K random molecules with conformations generated using RDKit, was employed. This dataset also provides a split into input fragments and a linker for each molecule.

In the second task, the models take the core part of the molecule, i.e., a scaffold, and adds side-chain specific motifs called R-groups. This molecule completion task is known as scaffold decoration. Usually, scaffold decoration is employed to enhance some molecular properties, for instance, binding affinity with a specific protein. A Multi R-Group Decoration Task on CrossDocked dataset, containing 100K ligand-protein pairs, where each ligand is split into a scaffold and R-groups, was used.

The PC transformer model takes molecular fragments/scaffold and protein pockets, if available, as an input point cloud and produces the linker/R-groups without repeating input atoms. The produced molecular substructures contain the attachment points described by a symbol ‘*’ and coordinates, as described above with respect to FIG. 8 (Block 880). These attachment points are utilized to combine input fragments with generated ones into a coherent molecule. The model can produce several R-groups in the scaffold decoration task. Each R-group also contains attachment points and is separated with a symbol ‘.’. See FIG. 16B for comparison of pocket-conditioned generation.

Comparisons of the PC transformer model (nach0-pc) with the DeLinker, 3DLinker, and DiffLinker models on the linker generation task, and against the LibINVENT, FLAG, and DiffDec models for scaffold decoration are discussed below.

As shown below in Table 3 and Table 4 for both tasks, the PC transformer model can complete input molecular point clouds with a high success rate, producing molecules that pass 2D filters such as PAINS. Despite moderate structural diversity, the PC transformer model produces spatially diverse molecules. Moreover, it enhances scaffold binding affinity, working on par with other currently state-of-the-art models in scaffold decoration.

TABLE 3
Models performance evaluation for linker design
task (metrics for baselines from [41]).

| | | | | | | |
|---|---|---|---|---|---|---|
| | | | 2D | | | |
| | Validity | Unique | Filters | Recovery | RMSD | SC_{RDKit} |
| Method | (↑) | (↑) | (↑) | (↑) | (↓) | (↑) |

State-of-the-art

| | | | | | | |
|---|---|---|---|---|---|---|
| DeLinker | 98.3 | 44.2 | 84.88 | 80.2 | 5.48 | 0.49 |
| [55] | | | | | | |
| 3DLinker | 71.5 | 29.2 | 83.72 | 93.5 | 0.11 | 0.92 |
| [56] | | | | | | |
| DiffLinker | 93.8 | 24.0 | 86.26 | 82.0 | 0.34 | 0.93 |
| [41] | | | | | | |

PC Transformer

| | | | | | | |
|---|---|---|---|---|---|---|
| Multi-Task | 81.6 | 27.6 | 99.00 | 50.5 | 1.28 | 0.86 |
| Single-Task | 89.7 | 12.3 | 99.55 | 36.5 | 1.04 | 0.88 |

### Shape-Conditioned Generation

Shape-conditioned generation focuses on producing molecules spatially similar to the reference structure but structurally dissimilar. This is achieved by representing the reference molecule as a shape—an area where molecular atom nucleus and electron clouds are located. In practice, molecules with similar shapes are more likely to have similar properties in drug discovery tasks, even when the structures are chemically distinguished. The molecular shape is represented as a point cloud. Each atom is replicated several times and Gaussian noise is added with predefined standard deviation σ to atom positions while removing all point features completely. Balance between spatial and chemical similarity can be achieved by alternating the parameter σ.

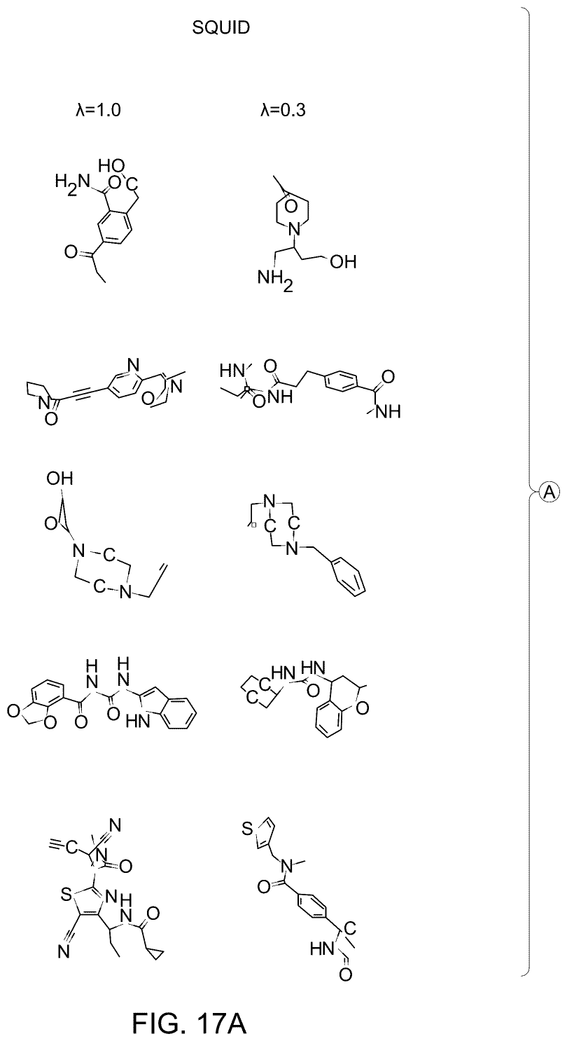

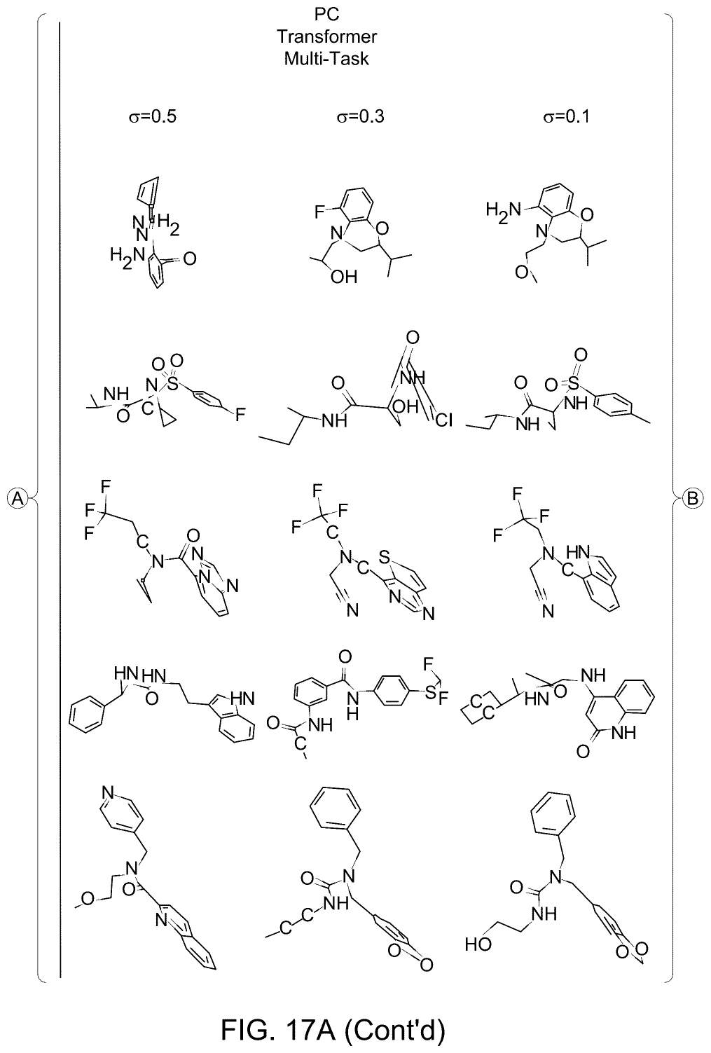

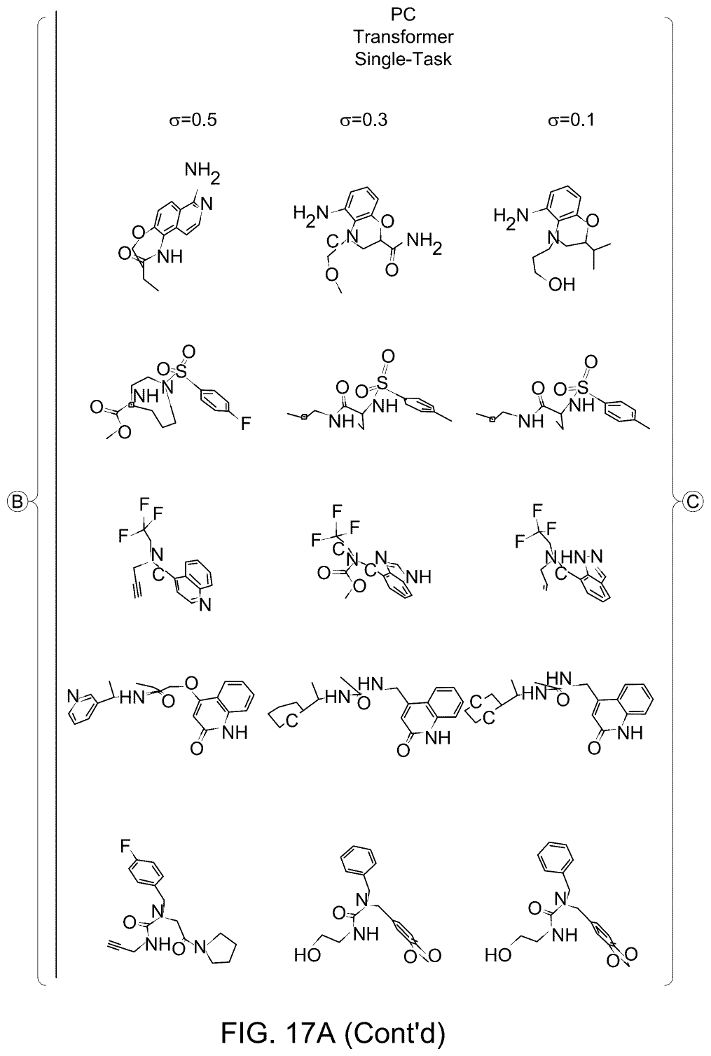

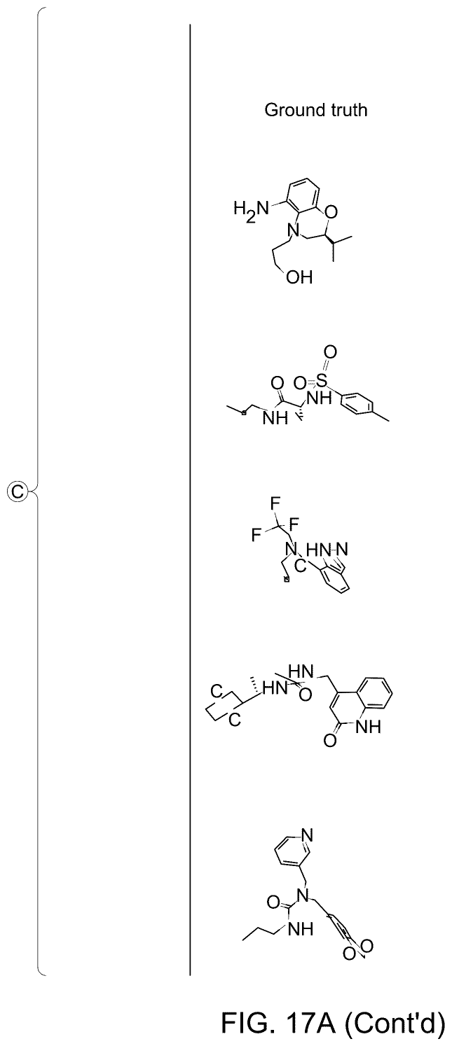

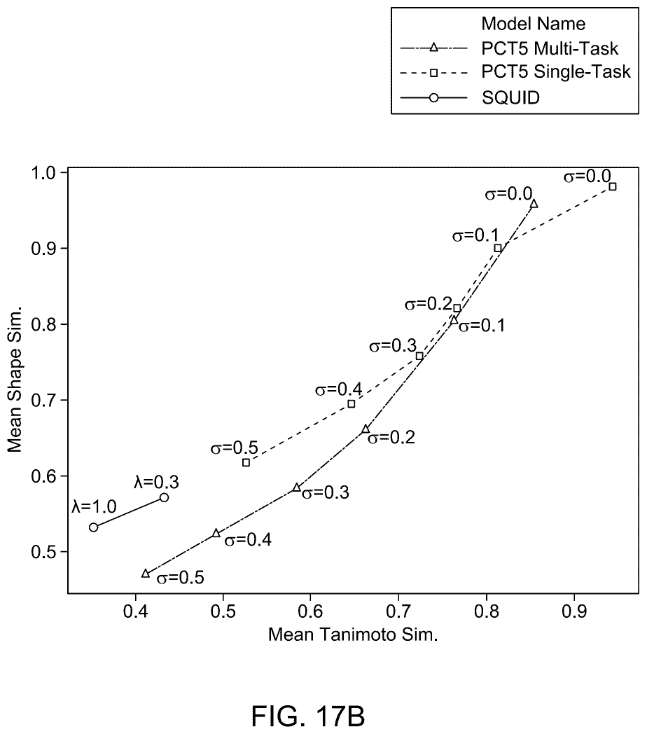

Training and test-stage sampling was conducted using the conformations computed with RDKit from the MOSES dataset. FIGS. 17A & 17B show a comparison of the PC transformer model with the SQUID model. Noise injection parameters were alternated, standard deviation σ for the PC transformer model (nach0-pc) and prior interpolation coefficient λ for SQUID, to show available trade-offs between structural and shape similarity. The PC transformer model is shown to provide a wider range of available trade-offs, covering a high structural to high shape similarity area. Moreover, the PC transformer model is shown to produce more spatially similar objects for low structural similarity values than SQUID.

### Pocket-Conditioned Generation

For pocket-conditioned generation, the PC transformer model was trained to generate novel high-affinity structures for a given protein pocket. The CrossDocked dataset was used and retained only high-affinity ligand-protein pairs. One hundred (100) molecules were randomly sampled during testing for each protein pocket in the test set. The generation quality of the PC transformer model (nach0-pc) was assessed. The comparison with the AR, Pocket2Mol and TargetDiff models is provided in Table 4.

TABLE 4
(left) Pocket-conditioned generation performance metrics (metrics of baselines)
(right) Scaffold decoration task metrics (metrics of baselines).

| | Valid- | Diver- | Vina | | High | | | | |
|---|---|---|---|---|---|---|---|---|---|
| | ity | sity | Dock (↓) | | Affinity | | Valid- | Unique- | QVina |
| Model | (↑) | (↑) | Avg. | Med | (↑) | Model | ity | ness | Score |
| Reference | 100% | — | −7.45 | −7.26 | — | Reference | — | — | −8.47 |
| | | | | | | Scaffolds | — | — | −7.73 |
| State-of-the-art | | | | | | State-of-the-art | | | |
| AR [63] | 92.95% | 0.70 | −6.75 | −6.62 | 37.9% | Pocket2Mol | 51.14% | 44.27% | −8.11 |
| | | | | | | [64] | | | |
| Pocket2Mol | 98.31% | 0.69 | −7.15 | −6.79 | 48.4% | FLAG [59] | 87.95% | 65.30% | −7.62 |
| [64] | | | | | | | | | |
| TargetDiff | 90.36% | 0.72 | −7.80 | −7.91 | 58.1% | DiffDec | 98.00% | 48.54% | −8.25 |
| [46] | | | | | | [43] | | | |
| PC Transformer | | | | | | PC Transformer | | | |
| Multi-Task | 91.78% | 0.32 | −6.52 | −6.86 | 38.2% | Multi-Task | 97.63% | 42.58% | −7.931 |
| Single-Task | 89.82% | 0.40 | −6.50 | −6.62 | 41.1% | Single-Task | 99.17% | 18.12% | −8.123 |

While there is a gap between the PC transformer model and the TargetDiff model in performance, the PC transformer model (nach0-pc) demonstrates comparable results to AR and Pocket2Mol based on docking scores and binding affinity.

Therefore, the PC transformer model (nach0-pc) is adept at generating diverse and physically plausible chemical 3D structures. By combining a domain-specific point cloud encoder with an encoder-decoder language model, the PC transformer model (nach0-pc) effectively addresses challenges associated with handling chemical 3D structures and SMILES sequences. Through extensive fine-tuning within single-task and multitask frameworks, the PC transformer model (nach0-pc) exhibits superior or comparable performance relative to various currently state-of-the-art diffusion models.

In some embodiments, the substance is a molecule, such as a small molecule, macromolecule, polypeptide, protein, antibody, oligonucleotide, nucleic acid (e.g., RNA, DNA, etc.), polypeptide, carbohydrate, lipid, or combinations thereof, whether natural or synthetic.

In some embodiments, the molecules that are generated are analyzed, and one or more specific molecules that fit specified condition criteria are selected. The selected one or more molecules are then selected and synthesized before being tested with one or more cells to determine whether or not the synthesized molecules actually satisfy the condition.

Once one or more molecules are generated, the model can categorize the molecules according to whatever profile is desirable. A specific physical property, such as certain chemical moieties or 3D structure can be prioritized, and then a molecule with a profile that matches the desired profile is selected and synthesized. As such, an object selector (e.g., molecule selector), which can be a software module, selects at least one molecule for synthesis, which can be done by filtering as described herein. The selected molecule is then provided to an object synthesizer, where the selected object (e.g., selected molecule) is then synthesized. The synthesized object (e.g., molecule) is then provided to the object validator (e.g., molecule validator), which tests the object to see if it satisfies the condition or property, or to see if it is biologically active for a specific use. For example, a synthesized object that is a molecule can be tested with live cell cultures or other validation techniques in order to validate that the synthesized molecule satisfies the desired property. As such, the objects, such as molecules or others, have real world versions that are obtained, such as by purchase, preparation, synthesis, or otherwise acquiring the physical form of the object.

Once a generated object is selected, then the method includes validating the selected object. The validation can be performed as described herein. When the object is a molecule, the validation can include synthesis and then testing with live cells.

In some embodiments, a method can include selecting a selected substance that corresponds with the selected generated data or that corresponds with the desired properties; and validating the selected substance. In some embodiments, the method may include: obtaining a physical version for the selected substance; and testing the physical version to have a desired property or biological activity. Also, in any method the obtaining of the physical version of the substance can include at least one of synthesizing, purchasing, extracting, refining, deriving, or otherwise obtaining the physical substance. The physical substance may be a molecule or other. The methods may include the testing involving assaying the physical substance in a cell culture. The methods may also include assaying the physical substance by genotyping, transcriptome-typing, 3-D mapping, ligand-receptor docking, before and after perturbations, initial state analysis, final state analysis, or combinations thereof. Preparing the physical version for the selected generated substance can often include synthesis when the physical substance is a new molecular entity. Accordingly, the methods may include selecting a generated object that is not part of the original dataset or previously known.

In some embodiments, the method can include: the obtaining of the physical form of the selected compound includes at least one of synthesizing, purchasing, extracting, refining, deriving, or otherwise obtaining the physical object; and/or the testing includes assaying the physical form of the selected object in a cell culture; and/or assaying the physical form of the selected compound by genotyping, transcriptome-typing, 3-D mapping, ligand-receptor docking, before and after perturbations, initial state analysis, final state analysis, or combinations thereof.

One skilled in the art will appreciate that, for the processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In one embodiment, the present methods can include aspects performed on a computing system. As such, the computing system can include a memory device that has the computer-executable instructions for performing the methods. The computer-executable instructions can be part of a computer program product that includes one or more algorithms for performing any of the methods of any of the claims.

In one embodiment, any of the operations, processes, or methods, described herein can be performed or cause to be performed in response to execution of computer-readable instructions stored on a computer-readable medium and executable by one or more processors. The computer-readable instructions can be executed by a processor of a wide range of computing systems from desktop computing systems, portable computing systems, tablet computing systems, hand-held computing systems, as well as network elements, and/or any other computing device. The computer readable medium is not transitory. The computer readable medium is a physical medium having the computer-readable instructions stored therein so as to be physically readable from the physical medium by the computer/processor.

There are various vehicles by which processes and/or systems and/or other technologies described herein can be affected (e.g., hardware, software, and/or firmware), and that the preferred vehicle may vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The various operations described herein can be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware are possible in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a physical signal bearing medium include, but are not limited to, the following: a recordable type of medium such as a floppy disk, a hard disk drive (HDD), a compact disc (CD), a digital versatile disc (DVD), a digital tape, a computer memory, or any other physical medium that is not transitory or a transmission. Examples of physical media having computer-readable instructions omit transitory or transmission type media such as a digital and/or an analog communication medium (e.g., a fiber optic cable, a waveguide, a wired communication link, a wireless communication link, etc.).

It is common to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. A typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems, including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. Such depicted architectures are merely exemplary, and that in fact, many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include, but are not limited to: physically mateable and/or physically interacting components and/or wirelessly interactable and/or wirelessly interacting components and/or logically interacting and/or logically interactable components.

FIG. 18 shows an example computing device 600 (e.g., a computer) that may be arranged in some embodiments to perform the methods (or portions thereof) described herein. In a very basic configuration 602, computing device 600 generally includes one or more processors 604 and a system memory 606. A memory bus 608 may be used for communicating between processor 604 and system memory 606.

Depending on the desired configuration, processor 604 may be of any type including, but not limited to: a microprocessor (μP), a microcontroller (μC), a digital signal processor (DSP), or any combination thereof. Processor 604 may include one or more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations, memory controller 618 may be an internal part of processor 604.

Depending on the desired configuration, system memory 606 may be of any type including, but not limited to: volatile memory (such as RAM), non-volatile memory (such as ROM, flash memory, etc.), or any combination thereof. System memory 606 may include an operating system 620, one or more applications 622, and program data 624. Application 622 may include a determination application 626 that is arranged to perform the operations as described herein, including those described with respect to methods described herein. The determination application 626 can obtain data, such as pressure, flow rate, and/or temperature, and then determine a change to the system to change the pressure, flow rate, and/or temperature.

Computing device 600 may have additional features or functionality, and additional interfaces to facilitate communications between basic configuration 602 and any required devices and interfaces. For example, a bus/interface controller 630 may be used to facilitate communications between basic configuration 602 and one or more data storage devices 632 via a storage interface bus 634. Data storage devices 632 may be removable storage devices 636, non-removable storage devices 638, or a combination thereof. Examples of removable storage and non-removable storage devices include: magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include: volatile and non-volatile, removable and non-removable media implemented in any method or technology for storage of information, such as computer readable instructions, data structures, program modules, or other data.

System memory 606, removable storage devices 636 and non-removable storage devices 638 are examples of computer storage media. Computer storage media includes, but is not limited to: RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 600 may also be implemented as a personal computer including both laptop computer and non-laptop computer configurations. The computing device 600 can also be any type of network computing device. The computing device 600 can also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general-purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. For example, one or more neural network configurations may be used to perform the specific features and acts described above.

In some embodiments, a computer program product can include a non-transient, tangible memory device having computer-executable instructions that when executed by a processor, cause performance of methods described herein. The non-transient, tangible memory device may also have other executable instructions for any of the methods or method steps described herein. Also, the instructions may be instructions to perform a non-computing task, such as synthesis of a molecule and or an experimental protocol for validating the molecule. Other executable instructions may also be provided.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.

### Examples

### A. Experimental Constraints

In this work, nach0 was utilized, which was pre-trained on SMILES data. A notable drawback of the SMILES format is the lack of a one-to-one correspondence between molecules and SMILES strings. A molecule can have multiple SMILES representations due to variations in the starting atom, molecular graph traversal, and kekulization. Second, scaling the PC transformer model for long chemical sequences may pose a challenge due to the quadratic nature of the attention mechanism. Finally, it is important to recognize that the academic datasets used in this study mainly include existing drugs and known chemical probes, covering only a small portion of the vast predicted chemical space. Moreover, they do not account for testing on novel chemical diversity that differs from molecules reported in the literature.

### B. Dataset Statistics and Examples of Inputs and Outputs

Several datasets were employed to evaluate and benchmark our generative models for different molecular generation tasks. These datasets provide comprehensive and high-quality molecular data, facilitating tasks such as property prediction, conformation generation, and drug design. Below are detailed descriptions of the datasets used. Table 5 shows examples of inputs and outputs for each task.

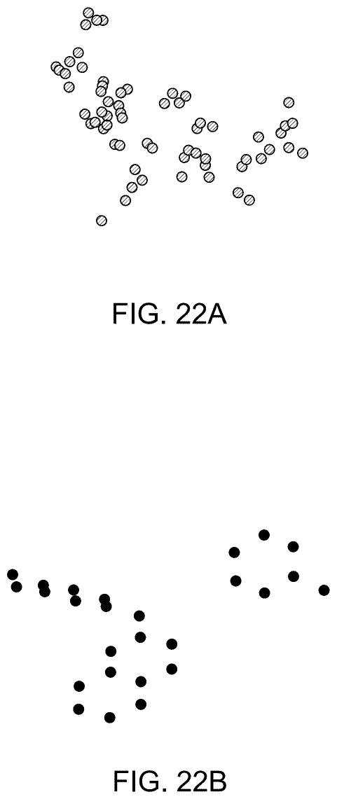

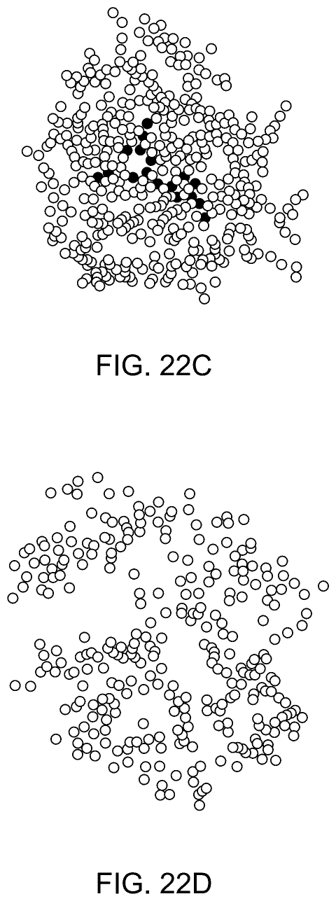

TABLE 5

| Task name | Input text | Input point cloud | Output text |
|---|---|---|---|
| Distribution | Generate molecular | No input PC | c1(c(ccc(Br)c1)O)C#N \| |
| learning | 3d structure from | | C −0.04 0.53 −0.51 \| |
| | GEOM | | C −1.24 0.22 0.15 \| |
| | | | C −1.23 −0.77 1.13 \| |
| | | | C −0.07 −1.44 1.45 \| |
| | | | C 1.11 −1.12 0.78 \| |
| | | | Br 2.71 −2.05 1.23 \| |
| | | | C 1.13 −0.15 −0.19 \| |
| | | | O −2.36 0.90 −0.19 |
| | | | C −0.02 1.53 −1.51 \| |
| | | | N 0.02 2.34 −2.33 |
| Conformation | Generate molecular | No input PC | S −0.39 −2.50 0.33 \| |
| generation | 3d structure for | | C 0.03 −0.90 0.02 |
| | S═c1ccccn1[O—] | | C 1.19 −0.56 −0.69 \| |
| | | | C 1.54 0.74 −0.95 \| |
| | | | C 0.71 1.76 −0.49 \| |
| | | | C −0.43 1.41 0.20 \| |
| | | | N −0.79 0.14 0.46 \| |
| | | | O −1.86 −0.09 1.11 |
| Shape- | Generate molecular | FIG. 22A | C(O)c1cc(c(c(Br)c1)OC)Br \| |
| conditioned | 3d structure for | | C −2.29 1.45 −1.64 \| |
| generation | shape | | O −1.74 2.64 −2.19 \| |
| | | | C −1.24 0.71 −0.86 \| |
| | | | C −1.57 0.06 0.34 |
| | | | C −0.60 −0.66 1.05 \| |
| | | | C 0.71 −0.73 0.58 \| |
| | | | C 1.04 −0.13 −0.64 \| |
| | | | Br 2.77 −0.28 −1.41 \| |
| | | | C 0.07 0.59 −1.35 \| |
| | | | O 1.66 −1.46 1.27 \| |
| | | | C 2.35 −0.63 2.20 \| |
| | | | Br −1.15 −1.55 2.64 |
| Linker design | Generate linker | FIG. 22B | *NC(═O)* \| |
| | | | * 3.17 1.82 −0.34 \| |
| | | | N 2.05 1.00 −0.70 \| |
| | | | C 2.01 −0.43 −0.56 \| |
| | | | O 3.07 −1.08 −0.33 \| |
| | | | * 0.75 −1.17 −0.76 |
| Scaffold | Generate decoration | FIG. 22C | C(C)*.C(C)* \| |
| decoration | | | C 2.50 0.39 1.83 \| |
| | | | C 3.94 0.49 2.38 \| |
| | | | * 2.38 −0.78 0.88 \| |
| | | | C 0.66 −3.60 −1.12 \| |
| | | | C 0.30 −4.67 −0.27 \| |
| | | | * 1.58 −2.55 −0.43 |
| Pocket- | Generate molecular | FIG. 22D | C1═NC(c2ccccc2)C═N1 \| |
| conditioned | 3d structure for | | C −2.54 1.46 −1.93 \| |
| generation | pocket | | N −1.96 0.35 −1.33 \| |
| | | | C −0.73 0.73 −0.94 \| |
| | | | C 0.25 −0.18 −0.24 \| |
| | | | C 0.44 −1.48 −0.68 \| |
| | | | C 1.35 −2.37 −0.04 \| |
| | | | C 2.08 −1.87 1.08 \| |
| | | | C 1.91 −0.55 1.56 \| |
| | | | C 0.98 0.33 0.88 \| |
| | | | C −0.62 2.01 −1.30 \| |
| | | | N −1.74 2.39 −1.89 |

“No input PC” indicates the absence of an input point cloud. Listed datasets were utilized in the pretraining stage. Imbalanced dataset sizes were dealt with by utilizing a balanced batch construction procedure, which includes the samples from different tasks with the same probability of forming the final training batch.

### B.1 GEOM

For spatial molecular distribution learning and conformation generation tasks, the GEOM-Drugs dataset was utilized, which provides conformer ensembles generated with metadynamics in CREST. The Geometric Ensemble of Molecules (GEOM) dataset is an extensive collection of molecular conformations generated using advanced sampling and semiempirical density functional theory (DFT). The dataset contains 37 million molecular conformations for over 450,000 molecules. The GEOM dataset includes conformers for 133,000 species from QM9 and 317,000 species with experimental data related to biophysics, physiology, and physical chemistry. The dataset also contains ensembles of 1,511 species with BACE-1 inhibition data labeled with high-quality DFT free energies in an implicit water solvent and 534 ensembles are further optimized with DFT. The dataset can be used for training and benchmarking models in tasks such as molecular property prediction, conformation generation, and drug design.

### B.2 ZINC Dataset

A subset of the ZINC dataset was utilized for the linker design task, consisting of 250,000 randomly selected molecules with low-energy conformations generated using RDKit. The ZINC dataset is a comprehensive collection of commercially available chemical compounds, primarily curated for virtual screening applications. It contains molecular graphs, each with up to 38 heavy atoms. This dataset is extensively used in computational chemistry and drug discovery research for tasks such as molecular property prediction. It provides a diverse and well-characterized set of molecules, facilitating the development of models that produce chemically valid, unique, and novel compounds with desirable properties.

Each molecule undergoes fragmentation by breaking all double cuts of acyclic single bonds, and the resulting splits are filtered according to a set of predefined rules. As a result of this fragmentation process, one molecule can yield various combinations of two fragments with a linker in between. In the experiments, the focus was on generating the linker without specifying anchor points (connecting points in the input fragment).

### B.3 MOSES

The MOSES dataset was utilized for the shape-conditioned generation task. Initially, this dataset is filtered to retain only molecules containing the 100 most prevalent fragments, and then 3D conformers are generated for the remaining molecules using RDKit. The MOSES dataset is a benchmarking platform that offers an extensive dataset and a suite of metrics for evaluating generative models in the context of unconditional molecular generation tasks. The dataset includes nearly 2 million molecular samples, filtered using MCF, PAINS, and other rules to ensure quality. The provided metrics assess the performance of generative models from multiple perspectives: the validity of generated structures, the quality of molecular distribution matching, and the models' capacity to generate novel and diverse molecules.

### B.4 CrossDocked

For the scaffold-decoration and pocket-conditioned generation tasks, the CrossDocked dataset was employed. It comprises 22.5 million protein-ligand complexes. It was preprocessed by leaving only 100,000 ligand-protein pairs with the highest binding affinity and clipping protein pocket region within 10 Å of ligand. Further, the LibINVENT slicing method with 37 customized reaction-based rules is applied on ligands to obtain scaffolds and R-groups.

### C. Evaluation Metrics

### C.1 Molecular Distribution Learning

The valid and complete molecules were evaluated from four perspectives: drug-likeness, 3D structures, bonds, and rings.

The drug-likeness of the generated molecules was assessed using the following metrics: (1) QED, which stands for quantitative estimation of drug-likeness; (2) SA, representing the synthetic accessibility score (higher values indicate easier synthesis); and (3) Lipinski, which measures the number of Lipinski's rule of five criteria the molecule meets.

A key distinction between 3D generation and 2D molecule graph generation is the determination of atom positions, making it essential to measure their accuracy. The minimal RMSDs were computed between the generated 3D molecules and 100 potential conformations predicted by the RDKit toolkit. Next, the most frequent bonds, bond pairs, and bond triplets were identified in the validation set. The bond lengths, bond angles, and dihedral angles were measured for both the generated molecules and those in the validation set. The Jensen-Shannon (JS) divergence was used to quantify the differences in the distributions between the generated molecules and the validation set.

The bond-related properties of the generated molecules were examined. Initially, the distribution of bond counts per atom were compared between the generated molecules and the validation set to determine if the models produced an excessive or insufficient number of bonds. Next, the distributions of various bond types were analyzed, including basic bond types (single, double, triple, and aromatic bonds) and the frequent bond types, bond pairs, and bond triplets used in the 3D structure evaluation.

The bond analysis was extended to include rings. First, the distribution of ring counts per molecule was compared between the generated molecules and the validation set. The distributions of counts for rings of various sizes (n-sized rings) were compared between the generated molecules and the validation set using JS divergence, averaging the JS divergence for n∈{3, 4, . . . , 9}.

### C.2 Conformation Generation

Evaluations were performed on the GEOM dataset, which offers conformer ensembles generated using metadynamics in CREST. As evaluation metrics for conformer generation, an approach was adopted utilizing Average Minimum RMSD (AMR) and Coverage (COV) for Precision (P) and Recall (R) measured when generating twice as many conformers as provided by CREST. For K=2L, let {C*ₗ}_{l∈[1,L]} and {Cₖ}_{k∈[1,K]} represent the sets of ground truth and generated conformers, respectively:

$$COV - R := \frac{1}{L} \vert \{ l ∈ [ 1 … L ] : ∃ k ∈ [ 1 … K ] , RMSD ( {C}_{k} , {C}_{l}^{*} ) < δ \} \vert$$

$$AMR - R := \frac{1}{L} {\sum }_{l ∈ [ 1 … L ]} {min}_{k ∈ [ 1 … K ]} RMSD ( {C}_{k} , {C}_{l}^{*} )$$

where δ is the coverage threshold. Following TorDiff, utilize δ=0.75 Å. Precision metrics are obtained by swapping the ground truth and generated conformers.

### C.3 Linker Generation

The Validity, Uniqueness, Recovery, RMSD, and SCRDKit of the samples was computed. Subsequently, it was ascertained whether the generated linkers adhere to the 2D filters utilized in generating the ZINC training set.

Validity—implement sanitization and additionally verify that the molecule includes all atoms from the fragments. For all other metrics, only a subset of valid samples were considered.

Uniqueness—compare SMILES of whole molecules and count the number of unique molecules sampled for each input pair of fragments.

Recovery—compare SMILES of each molecule sampled for a given pair of fragments with SMILES of the corresponding ground-truth molecule. Before comparison, hydrogens and stereochemistry were removed from molecules.

RMSD—To evaluate the 3D conformations of the sampled versus ground-truth molecules, determine the Root Mean Squared Deviation (RMSD) between the coordinates of the generated linkers and those of the real molecules in cases where the original molecules are accurately recovered. To compute RMSD, only recovered molecules were considered and aligned with the corresponding ground-truth molecules using RDKit function rdkit. Chem.rdMolAlign, which returns the optimal RMSD for aligning two molecules.

The SCRDKit metric was utilized to measure both the geometric and chemical similarities between the generated molecules and the ground-truth counterparts.

2D filters were applied, including synthetic accessibility, ring aromaticity (RA), and pan-assay interference compounds (PAINS), to create the ZINC and CASF datasets. The RA filter ensures correct covalent bond orders in ring structures, while the PAINS filter detects compounds prone to producing false-positive results in high-throughput screenings.

### C.4 Scaffold Decoration

The same metrics were employed for scaffold decoration as for linker generation. Additionally, the Vina Scores and High Affinity metrics were incorporated.

Vina scores are calculated by QVina software to measure the binding affinity. QVina is designed to perform the same function as AutoDock Vina—predicting the binding affinity and orientation of a ligand to a protein receptor—but with optimizations that allow it to perform these calculations more quickly. The Vina scores serve as a metrics to evaluate the binding affinity between a ligand and a receptor in docking simulations. It quantitatively represents the predicted binding energy, with lower scores indicating stronger binding affinity. This score is used in assessing the likelihood of successful ligand-receptor interactions and is particularly valuable in computational drug discovery and virtual screening efforts.

### C.5 Shape-Conditioned Generation

To evaluate shape-conditioned generation tasks, graph similarity (SimG) and shape similarity (SimS) metrics were utilized.

SimG was utilized to define graph (chemical) similarity, simG∈[0,1], between two molecules as the Tanimoto similarity, computed by RDKit with default settings using 2048-bit fingerprints.

SimS was utilized to shape similarity metric from ESP-Sim package.

### C.6 Pocket-Conditioned Generation

Vina Dock was utilized the pocket-conditioned generation tasks. To calculate the Vina Dock values, AutoDock Vina, a widely employed software for molecular docking studies, was utilized.

The High Affinity metric was used to measure how strongly a ligand binds to a receptor in molecular docking or biochemical assays in comparison with reference ligand. It helps predict successful interactions between molecules. It considers factors like the stability of the ligand-receptor complex, the strength of interactions between them (like hydrogen bonds), and the overall energy of the interaction. Vina Dock values were compared for generated and reference molecules and provide percentage of cases when generated molecules binds better than reference molecules.

### D. Model Parameters and Training Details

A model based on the nach0 architecture was utilized. The experiments involved a base model variant, containing 370M parameters, characterized by 12 layers, a hidden state of 768 dimensions, a feed-forward hidden state of 2048 dimensions, and 12 attention heads.

For the selected model, pre-training was conducted with a language modeling (LM) objective and subsequent fine-tuning. The base model was trained using two NVIDIA A6000 GPUs. The probability of molecular fragment dropout on the pretraining stage is 0.2.

The pre-training and fine-tuning stages were executed using the following hyperparameters: a batch size of 64 for both pre-training and fine-tuning, a learning rate set to 1e-4, a weight decay of 0.01, and a cosine schedule. The pre-training stage lasted for 100000 steps, whereas the fine-tuning stage also for 100000 steps.

### E. Additional Related Work

### E.1 Linker Generation

Three models have emerged as significant contributors to linker generation: DeLinker, 3DLinker, and DiffLinker.

DeLinker is a model adapted for linker design. It particularly retains the 3D structural information and generates linkers by providing two input fragments. It is one of the first attempts to apply Graph Neural Networks (GNN) in linker design.

3DLinker is an E(3) Equivariant Variational Autoencoder for Molecular Linker Design. It generates a small “linker” to physically attach two independent molecules with their distinct functions. The generation of linkers is conditional on the two given molecules, and the linkers heavily depend on the anchor atoms of the two molecules to be connected. It predicts anchor atoms and jointly generates linker graphs and their 3D structures3.

DiffLinker is an Equivariant 3D-conditional Diffusion Model for Molecular Linker Design. Given a set of disconnected fragments in 3D, DiffLinker places missing atoms in between and designs a molecule incorporating all the initial fragments. Unlike previous approaches, which can only connect pairs of molecular fragments, DiffLinker can link an arbitrary number of fragments.

### E.2 LLaMa Baseline

For establishing a comparative single-domain baseline the model and tokenizer were trained from scratch with LLaMa-like architecture. For both cases, the implementations of Open LLaMa from the HuggingFace framework were adopted and training was performed on the GEOM dataset.

In this study, the Tokenizer remains consistent with the Open LLaMa version, though it was retrained solely on recent datasets. No improvements were made to the tokenization process for digits or molecules and restricted the vocabulary size to 512 tokens.

Concerning the model, modifications had been applied to its configuration to align with the dimensions of nach0-pc. Namely, the number of hidden layers and attention heads were set to 12, hidden size to 768, and intermediate size to 2048. The overall parameter count of the model reached nearly 90M. During the training procedure, the global batch size was set to be equal to 32, the learning rate to 1e-4 with a linear scheduler with warm-up steps, and weight decay to 1e-2. The model was trained using the causal language modeling objective for 10 epochs.

### E.3 Attention-Based Models for Point Clouds

Recently, there have been several investigations into applying the Transformer architecture to point cloud analysis. The Point Cloud Transformer (PCT) [13] utilizes a permutation-invariant transformer instead of a self-attention mechanism to manage unstructured and disordered point data in irregular domains. Similarly, a Transformer-based network (TR-Net) PointConT [71] leverages the locality of points in the feature space by clustering sampled points with similar features into the same class and computing self-attention within each class. The design is intended to capture long-range dependencies within the point cloud while maintaining computational efficiency. [72] employs a neighborhood embedding strategy along with a residual backbone featuring skip connections to enhance context aware and spatial-aware features. The network utilizes an offset attention operator on point cloud spatial information to refine attention weights, thereby improving the extraction of global features.

### E.4 Non-Diffusion Approaches.

Several neural generative models were proposed to generate spatial molecular structures, including generators working directly with atomic density grid and voxels. A conditional variational autoencoder [73] was trained on atomic density grid representations of cross-docked protein-ligand structures. To construct valid molecular conformations from the generated atomic densities, atom fitting and bond inference procedures were utilized. Pocket2Mol [64], an E(3)-equivariant generative network, consists of two modules: 1) a graph neural network (GNN) that captures both spatial and bonding relationships between atoms in the binding pockets, and 2) an algorithm that samples drug candidates based on pocket representations from a tractable distribution. VoxMol [74] samples noisy density grids from a smooth distribution using underdamped Langevin Markov chain Monte Carlo and denoises the noisy grid in a single step to refine the exact atom positions. Unlike point-cloud diffusion models, VoxMol is simpler to train, does not require prior knowledge of the number of atoms, and does not treat features as different distributions.

### F. Computational Resources

### F.1 Hardware Computational Resources

The PC transformer (nach0-pc) model was utilized for the various experiments, leveraging state-of-the-art computational hardware to ensure efficient and effective training and evaluation. The hardware configuration of the CoreWeave cloud service provider included:

GPU: 2*NVIDIA RTX A6000 GPUs with 48 GB of memory for PC transformer model training and 2*RTX A4000 GPUs with 16 GB of memory for compared model training. These GPUs are specifically designed for deep learning tasks, providing high throughput and large memory capacity, which are essential for handling our model's extensive computational demands. CPU: The AMD EPYC 7413 processor has 24 cores and a base clock speed of 2.65 GHz, supporting multi-threaded operations and high parallelism. RAM: 128 GB of DDR4 memory to support large batch processing and extensive model parameter storage. Storage: High-speed NVMe SSDs with a total capacity of 1 TB to ensure rapid data access and model checkpointing.

### F.2 Training and Evaluation Time

Training and evaluating the PC transformer model required computational time as follows:

Training Time: The initial pre-training phase took 40 hours. This phase included preprocessing the dataset, training the model across multiple epochs, and hyperparameter tuning. Finetuning time: Finetuning phase took around 60 hours. Evaluation Time: The evaluation phase, which involved running inference, calculating performance metrics, and validating results, took an additional 6 hours (excluding the ablation study sampling). Multiple runs were conducted to verify the consistency of the results and ensure robustness and accuracy.

In summary, the deployment of the PC transformer model on advanced computational resources, although costly and time-consuming, was crucial for achieving high-performance results in our experiments. Investing in cutting-edge hardware facilitated efficient training and rigorous evaluation, underscoring the importance of adequate resources in modern machine learning research.

### G. Point Cloud Encoder Details and Visualizations

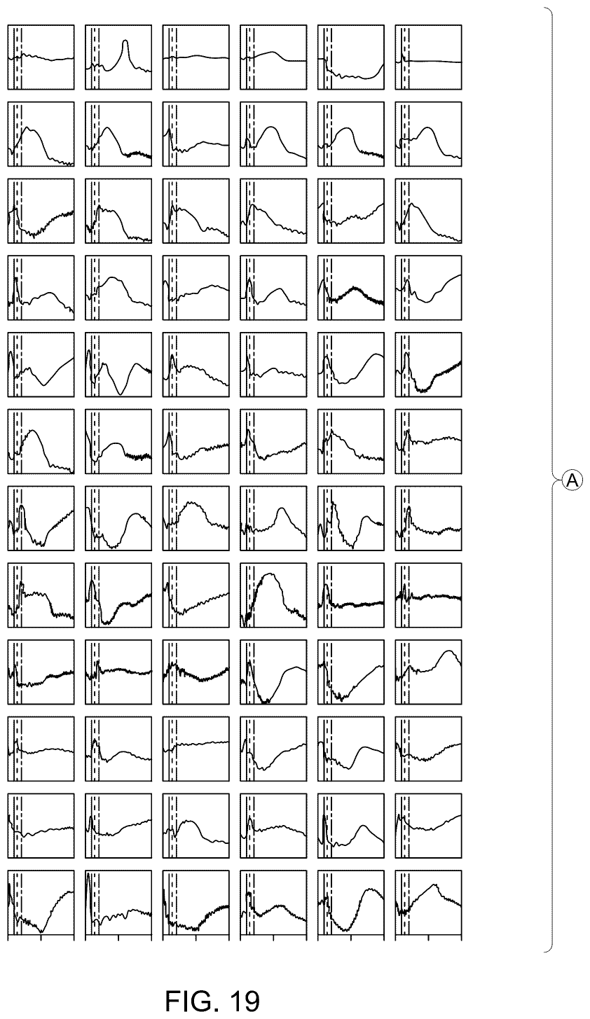

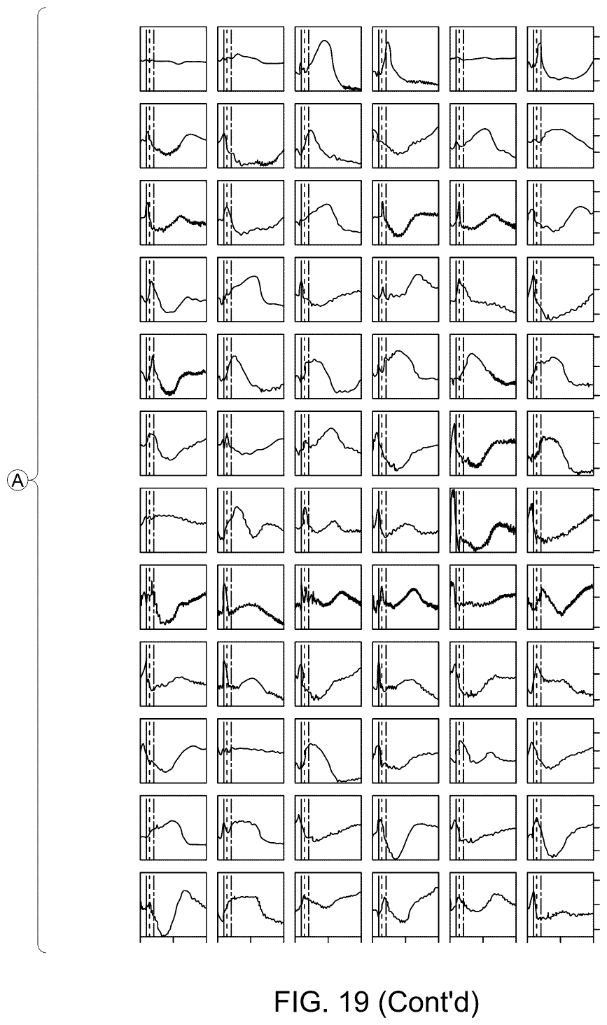

FIG. 19 illustrates a visualization of learned relative biases in accordance with an embodiment. The green lines correspond to a typical C—C single covalent bond. The yellow lines correspond to typical hydrogen bond length. The red lines correspond to the typical distance between consequent CA-CA atoms in the protein. As shown, the activation values of relative biases are illustrated as a function of the distances between atoms. On the X-axis, the distances are represented, ranging from 0 to 20 Å, while the Y-axis depicts the corresponding activation values, ranging from −25 bias to +25 bias for the top row of graphs and −10 bias to +10 bias for the rest of the rows of graphs. This visualization was conducted using synthetic data, where the distances between atom coordinates were systematically varied. The dashed lines indicate typical atomic distances: the green dashed line represents the typical length of a C—C single covalent bond, the yellow dashed line corresponds to the typical hydrogen bond length, and the red dashed line signifies the typical distance between consecutive CA-CA atoms in a protein. Notably, the activations in some attention heads within particular layers reach their peak values precisely in the regions corresponding to these typical bond lengths, highlighting the model's sensitivity to biologically relevant atomic distances.

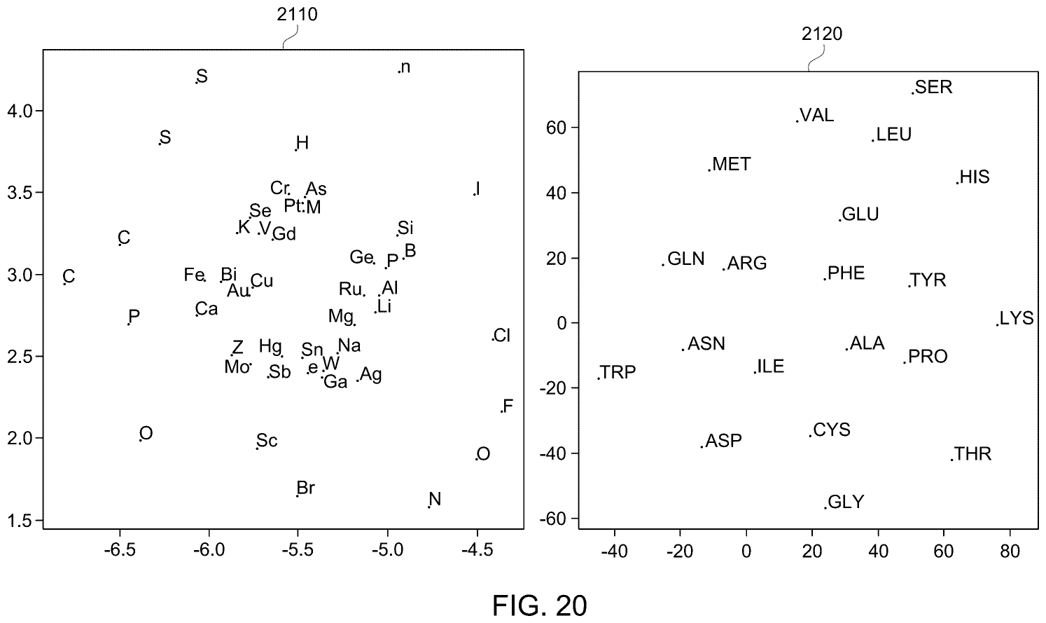

FIG. 20 illustrates t-SNE visualizations 2100 of textual atom token embeddings 2110 and textual amino acid token embeddings 2120. These embeddings are projected into a 2D space where similar tokens are positioned based on their common chemical and structural characteristics. For atom tokens 2110, the visualization highlights how atoms with similar properties or roles within molecules are represented. Similarly, the amino acid token embeddings 2120 reflect their structural similarities, demonstrating the model's ability to capture and encode essential chemical and structural information within the textual token embeddings.

### H. Analysis: Design Choices of the PC Transformer Model (Nach0-Pc)

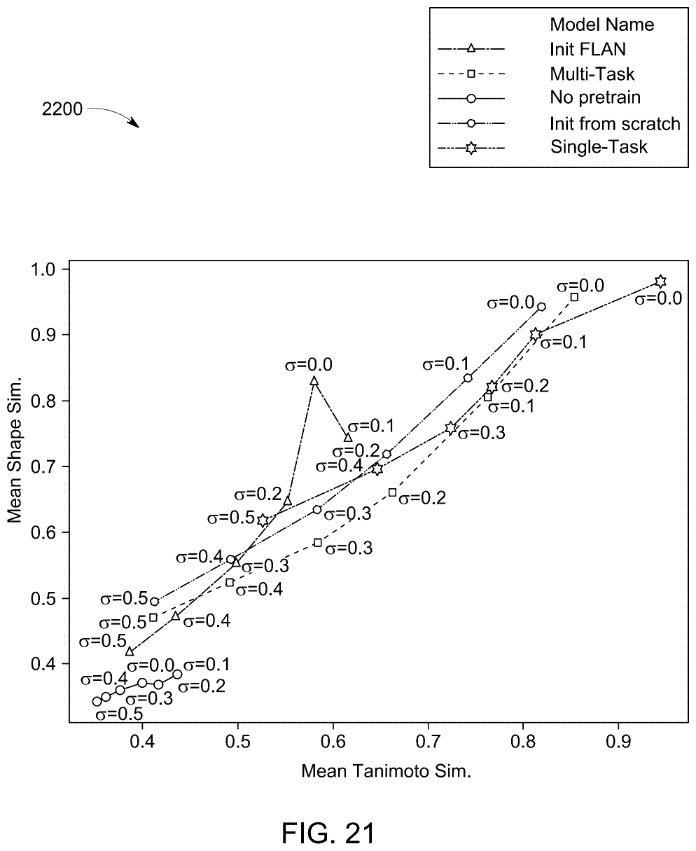

This section explores the role of the language model (LM) within the PC transformer model. It begins with selecting the LM component, where the PC transformer model is initialized with nach0, a top-tier chemical LM. Comparative analyses reveal that models leveraging nach0 outperform those using other LMs across various tasks, including distribution learning, conformation generation, linker design, and pocket-conditioned generation. Notably, nach0-based models consistently exhibit superior performance, underscoring the efficacy of domain-specific pre-training. This section provides more details of design choices of the PC transformer model on spatial molecular distribution learning (Tab. 6), conformation generation (Tab. 7), linker design (Tab. 8), shape-conditioned generation (FIG. 21) and pocket-conditioned generation (Tab. 9) tasks.

### H.1 LM Component

The first design choice of the PC transformer model is the LM component. For all experiments presented, the PC transformer model's LM component was initialized with nach0, a state-of-the-art chemical LM for multi-domain tasks. In this section, several LMs are compared: (i) the state-of-the-art cross-domain nach0, (ii) the general-domain FLAN, and (iii) random initialization.

As for the distribution learning task, several observations can be made from Table 6.

TABLE 6

| | | PC Transformer | | | | |
|---|---|---|---|---|---|---|
| | | | | | | No |
| | | Multi- | Single- | | From | pre- |
| Group | Metrics | Task | Task | FLAN | scratch | train |
| Druglikeness | QED (↑) | 0.770 | 0.664 | 0.634 | 0.712 | 0.828 |
| | SA (↑) | 0.872 | 0.848 | 0.825 | 0.858 | 0.871 |
| | Lipinski (↑) | 4.993 | 4.938 | 4.923 | 4.973 | 5.0 |
| 3D | JS. bond lengths (↓) | 0.205 | 0.146 | 0.397 | 0.190 | 0.326 |
| structures | JS. bond angles (↓) | 0.107 | 0.100 | 0.247 | 0.137 | 0.152 |
| | JS. dihedral | 0.133 | 0.113 | 0.229 | 0.129 | 0.188 |
| | angles (↓) | | | | | |
| Bonds | JS. num. bonds per | 0.230 | 0.094 | 0.181 | 0.159 | 0.537 |
| | atoms(↓) | | | | | |
| | JS. freq. bond | 0.050 | 0.033 | 0.067 | 0.061 | 0.095 |
| | types (↓) | | | | | |
| | JS. freq. bond | 0.038 | 0.033 | 0.101 | 0.040 | 0.056 |
| | pairs (↓) | | | | | |
| | JS. freq. bond | 0.054 | 0.041 | 0.123 | 0.043 | 0.085 |
| | triplets (↓) | | | | | |
| Rings | JS. num. rings (↓) | 0.267 | 0.036 | 0.079 | 0.154 | 0.492 |
| | JS. num. n-sized | | | | | |
| | rings (↓) | | | | | |
| | Num. Intersecting | 0.0599 | 0.0248 | 0.0456 | 0.0389 | 0.1026 |
| | rings (↑) | | | | | |
| Mean RMSD | Mean RMSD min (↓) | 1.087 | 1.347 | 1.492 | 1.259 | 0.832 |

First, the single-task and multi-task PC transformer model with nach0 outperformed other models on 3D Structures, Bond, and Ring Metrics. Second, the single-task model has the lowest JS divergence (0.146), followed by the multi-task model (0.205). The single-task model performs best (0.100 in the JS bond angles metric), while the multi-task model is close behind (0.107). The FLAN-based model shows the highest divergence (0.247). Overall, the single-task model consistently achieves the lowest JS divergences across various metrics, indicating high accuracy in bond and ring structures. Both the PC transformer model with nach0 and the random-based LM components outperform the general-domain LM component FLAN. This indicates that the PC transformer model with domain-specific nach0 provides significant improvements over the model with general-domain FLAN in downstream tasks in both single-task and multi-task settings.

Similar observations can be made for the conformation generation task. Table 7, a comparison of model performance on conformation generation, indicates that incorporating multi-tasking and domain-specific pre-training significantly enhances the quality metrics for the task of generating conformations.

TABLE 7

| | Recall | | | | Precision | | | |
|---|---|---|---|---|---|---|---|---|
| | COV (↑) | | MAT (↓) | | COV (↑) | | MAT (↓) | |
| Method | Mean | Med | Mean | Med | Mean | Med | Mean | Med |
| Multi-Task | 50.06 | 50.00 | 0.7609 | 0.7533 | 27.65 | 16.67 | 1.1098 | 1.1144 |
| Single-Task | 57.67 | 59.53 | 0.7054 | 0.6944 | 32.45 | 23.13 | 1.0314 | 1.0340 |

Another init checkpoints

| | | | | | | | | |
|---|---|---|---|---|---|---|---|---|
| FLAN | 38.68 | 26.67 | 0.8535 | 0.8499 | 19.99 | 8.33 | 1.2054 | 1.1889 |
| From scratch | 34.38 | 22.31 | 0.9152 | 0.9032 | 16.50 | 6.74 | 1.3891 | 1.3697 |

Input

| | | | | | | | | |
|---|---|---|---|---|---|---|---|---|
| Canonical SMILES | 44.19 | 39.52 | 0.8167 | 0.8148 | 25.90 | 14.29 | 1.1472 | 1.1477 |
| Non-isomeric | 50.41 | 47.30 | 0.7630 | 0.7625 | 27.32 | 16.67 | 1.1163 | 1.1206 |
| SMILES | | | | | | | | |

Pre-training

| | | | | | | | | |
|---|---|---|---|---|---|---|---|---|
| No pre-training | 57.99 | 60.63 | 0.7014 | 0.6945 | 34.20 | 25.00 | 1.0228 | 1.0237 |

The table evaluates the PC transformer model in multi-task and single-task settings with a nach0 backbone, as well as the performance with the FLAN and random LM component and no pre-train models.

In addition to the improvements observed in the task of generating conformations, Table 8 highlights the substantial benefits of incorporating multi-tasking and domain pre-training for the linker design task.

TABLE 8

| | | | 2D | | | |
|---|---|---|---|---|---|---|
| | Validity | Unique | Filters | Recovery | RMSD | SC_{RDKit} |
| Method | (↑) | (↑) | (↑) | (↑) | (↓) | (↑) |
| Multi-Task | 81.6 | 27.6 | 99.00 | 50.5 | 1.28 | 0.86 |
| Single-Task | 89.7 | 12.3 | 99.55 | 36.5 | 1.04 | 0.88 |

Another init checkpoints

| | | | | | | |
|---|---|---|---|---|---|---|
| FLAN | 81.1 | 30.9 | 98.97 | 54.25 | 1.25 | 0.86 |
| From | 83.9 | 24.1 | 99.20 | 56.0 | 1.17 | 0.87 |
| scratch | | | | | | |

Pre-training

| | | | | | | |
|---|---|---|---|---|---|---|
| No | 34.8 | 61.8 | 99.03 | 23.7 | 1.59 | 0.83 |
| pre-training | | | | | | |

Table 8 shows a comparison of model performance on linker design. The table evaluates the PC transformer model in multitask and single-task settings with a nach0 backbone, as well as the performance with the FLAN and random LM component and no pre-train models. Similar to the enhancements seen in conformation generation, the inclusion of multi-task learning and pre-training techniques significantly boosts the quality metrics for linker design.

Table 9 shows a comparison of model performance on pocket-conditioned generation tasks.

TABLE 9

| | Validity | Diversity | Vina Dock (↓) | | High |
|---|---|---|---|---|---|
| Model | (↑) | (↑) | Avg. | Med | Affinity (↑) |
| Multi- | 91.78% | 0.32 | −6.52 | −6.86 | 38.2% |
| Task | | | | | |
| Single- | 89.82% | 0.40 | −6.50 | −6.62 | 41.1% |
| Task | | | | | |

Another init checkpoints

| | | | | | |
|---|---|---|---|---|---|
| FLAN | 86.03% | 0.34 | −6.26 | −6.67 | 36.1% |
| From | 93.02% | 0.34 | −6.77 | −6.82 | 38.4% |
| scratch | | | | | |

Pre-training

| | | | | | |
|---|---|---|---|---|---|
| No pre- | 93.02% | 0.26 | −5.70 | −6.43 | 33.1% |
| training | | | | | |

The table evaluates the PC transformer model in multi-task and single-task settings with a nach0 backbone, as well as the performance with the FLAN and random LM component and no pre-train models. The better scores across all models are highlighted in bold.

Corresponding outcomes emerge when examining the results of the pocket-conditioned generation task. The integration of multi-tasking and pre-training methods leads to noticeable improvements in quality metrics.

Another way to compare the impact of implemented enhancements is to analyze the dependence curve, which delineates the relationship between Mean Tanimoto similarity and Mean shape similarity across various alpha values. A robust model is expected to yield lower Tanimoto similarities for the same levels of shape similarity. FIG. 21 illustrates a graphical comparison 2200 of model performance on shape-conditioned generation in accordance with an embodiment. The figure evaluates the PC transformer model in multi-task and single-task settings with a nach0 backbone, as well as the performance with the FLAN and random LM component and no pre-train models. Graphical comparison 2200 illustrates these curves for models with different levels of ablations. The comparison demonstrates that the multi-task model performs worse than the single-task model, as evidenced by its higher Tanimoto similarities for comparable shape similarities.

### H.2 Impact of 3D Pre-Training

Another significant contribution of the PC transformer model is its 3D pre-training approach. Taking into account the three fundamental aspects of machine learning—data and task complexity—pre-training is advantageous when the amount of data available for the downstream task is relatively small compared to the complexity of the task.

As shown in Table 6, the pre-trained and fine-tuned PC transformer model provides significant improvements over a fine-tuned the PC transformer model without pre-training on 3D Structures, Bond, and Ring Metrics. Surprisingly, model pre-training achieved better results than the single-task PC transformer model on the conformation generation task. As for the linked design task, similar observations can be made as on the distribution learning task: 3D pre-training helps the model perform the downstream task.

### H.3 Canonical and Non-Isometric SMILES

the PC transformer model augments isomeric SMILES that include stereo labels. This allows the model to utilize some 3D information present in stereo labels of isomeric SMILES and to generalize better with augmentation. Ablation studies were conducted to compare this choice against a non-augmented version of isomeric SMILES, which is called canonical SMILES in Table 7, as well as non-isomeric SMILES without augmentation. Table 7 shows that the main multi-task PC transformer model outperforms the canonical isomeric SMILES approach without augmentation on conformation generation but is on par with the non-augmented non-isomeric SMILES approach. The results suggest that augmentation boosts performance. However, a non-isomeric SMILES choice might also be an option for unconditional conformation generation with relatively large and diverse datasets.

### I. Model Training Time and CO2 Impact

Table 10 shows GPU computation time and CO2 emissions for the PC transformer model and state-of-the-art diffusion models.

TABLE 10

| | | | | | Total |
|---|---|---|---|---|---|
| | | | CO2 | Total | CO2 |
| | | Time | Emissions | time | Emissions |
| Model | Task | (↓) | (↓) | (↓) | (↓) |
| MolDiff | Training | 60 | 5.55 | 180 | 16.65 |
| | 10k Sampling | 120 | 11.1 | | |
| EDM | Training | 730 | 67.53 | 840 | 77.71 |
| | 10k Sampling | 110 | 10.18 | | |
| PC | Pre-train | 39.5 | 4.98 | 164.5 | 20.73 |
| Transformer | Finetune | 119 | 14.99 | | |
| | (multi-task, | | | | |
| | 2 GPU) | | | | |
| | Finetune | 59.5 | 7.5 | | |
| | (single task, | | | | |
| | 1 GPU) | | | | |
| | 10k Sampling | 6 | 0.76 | | |
| | Resources per 1 | — | — | 27.4 | 3.5 |
| | task | | | | |
| Tor. Diff. (*) | Training | 360 | 33.72 | 393 | 38.33 |
| | 10k Sampling | 33 | 4.61 | | |
| TargetDiff (*) | Training | 24 | 2.9 | 95 | 12.23 |
| | 10k Sampling | 71 | 9.33 | | |
| Pocket2Mol (*) | Training | 132 | 11.8 | 227 | 26 |
| | 10k Sampling | 95 | 14.2 | | |

In Table 10, timings (marked with *) are extracted from reference papers. Single-task fine-tuning was excluded from the total time and total CO2 emission for the PC transformer model and exists here only for the direct comparison purpose with single-task models.

As delineated in Table 10, the computational resources necessitated for the pre-training phase of the PC transformer model are quantified in GPU hours (total training duration). This encompasses the pre-training on the datasets, the fine-tuning of the model across all datasets, and the generation of molecules with the models.

Particularly, the fine-tuned models originating from the pre-trained checkpoints exhibit enhanced performance while consuming approximately half of the computational resources necessitated by the models trained ab initio. This efficient resource allocation when implementing the PC transformer model technique corroborates its feasibility. Moreover, the initial allocation of resources for pre-training the models is well warranted as these models, once trained, can be repurposed across many applications, thereby augmenting their utility and cost-effectiveness. All experiments were executed utilizing the CoreWeave infrastructure. For the described PC transformer model training and evaluation, a cumulative computation time of 164.5 hours was performed on Nvidia RTX A6000 48 GB hardware (with a TDP of 300 W). The total emissions for the model were estimated to be 20.73 kgCO2 eq. Comparing the PC transformer model with the other SOTA diffusion models discussed also took 1020 hours on the Nvidia RTX A4000 16 GB (with a TDP of 140 W) and 94.36 kgCO2 eq of emissions. These estimations were made using the Machine Learning Impact calculator.

The PC transformer model is multitasking, trained sequentially for all six tasks. As shown in Table 10, the PC transformer model showed effective results in computation times and CO2 emissions when compared directly to the other single-task models. According to the total time and CO2 emissions, divided by the number of tasks, the PC transformer model is 3.5 times more effective than the second most effective single-task model while maintaining acceptable quality in all six tasks.

The following datasets were used: 1) the GEOM dataset, 2) the MOSES dataset, 3) The ZINC dataset, and the CrossDocked dataset. The following models were used: 1) nach0, and 2) The FLAN model. To perform ablation studies, the following model architectures were used: 1) OpenLLaMa source, and 2) MolDiff model source code.

Cross-reference is made to the following incorporated references: U.S. Pat. Nos. 11,568,961; 11,403,521; US 2015/0178442; US 2020/0090049; US 2020/0082916; US 2020/0258594; US 2022/0310196; US 2021/0233621; US 2021/0271980; US 2021/0287067; US 2021/0383898; US 2022/0172802; US 2022/0406404; EP 3289501; WO 2021/165887; and WO 2021/229454.

The following references are incorporated herein by specific reference.

### REFERENCES

[1] Jacob Devlin, et al.; BERT: Pre-training of deep bidirectional transformers for language understanding. In Proceedings of the 2019 Conference of the North American Chapter of the Association for Computational Linguistics: Human Language Technologies, Volume 1 (Long and Short Papers), pages 4171-4186, Minneapolis, Minnesota, June 2019. Association for Computational Linguistics.
[2] Colin Raffel, et al. Exploring the limits of transfer learning with a unified text-to-text transformer. Journal of Machine Learning Research, 21(140): 1-67, 2020.
[3] Mike Lewis, et al.; BART: Denoising sequence-to-sequence pretraining for natural language generation, translation, and comprehension. In Dan Jurafsky, Joyce Chai, Natalie Schluter, and Joel Tetreault, editors, Proceedings of the 58th Annual Meeting of the Association for Computational Linguistics, pages 7871-7880, Online, July 2020. Association for Computational Linguistics.
[4] Zhangyin Feng, et al.; CodeBERT: A pre-trained model for programming and natural languages. In Trevor Cohn, Yulan He, and Yang Liu, editors, Findings of the Association for Computational Linguistics: EMNLP 2020, pages 1536-1547, Online, November 2020. Association for Computational Linguistics.
[5] Tom Brown, et al.; Language models are few-shot learners. In H. Larochelle, M. Ranzato, R. Hadsell, M. F. Balcan, and H. Lin, editors, Advances in Neural Information Processing Systems, volume 33, pages 1877-1901. Curran Associates, Inc., 2020.
[6] Jean-Baptiste Alayrac, et al.; Flamingo: a visual language model for few-shot learning. In S. Koyejo, S. Mohamed, A. Agarwal, D. Belgrave, K. Cho, and A. Oh, editors, Advances in Neural Information Processing Systems, volume 35, pages 23716-23736. Curran Associates, Inc., 2022.
[7] Jing Yu Koh, Ruslan Salakhutdinov, and Daniel Fried. Grounding language models to images for multimodal inputs and outputs. In Andreas Krause, Emma Brunskill, Kyunghyun Cho, Barbara Engelhardt, Sivan Sabato, and Jonathan Scarlett, editors, Proceedings of the 40th International Conference on Machine Learning, volume 202 of Proceedings of Machine Learning Research, pages 17283-17300. PMLR, 23-29 Jul. 2023.
[8] Jiasen Lu, Dhruv Batra, Devi Parikh, and Stefan Lee. VILBERT: Pretraining task-agnostic visiolinguistic representations for vision-and-language tasks. In H. Wallach, H. Larochelle, Beygelzimer, F. d'Alché-Buc, E. Fox, and R. Garnett, editors, Advances in Neural Information Processing Systems, volume 32. Curran Associates, Inc., 2019.
[9] Chen Sun, Austin Myers, Carl Vondrick, Kevin Murphy, and Cordelia Schmid. VideoBERT: A joint model for video and language representation learning. In 2019 IEEE/CVF International Conference on Computer Vision (ICCV), pages 7463-7472, Los Alamitos, CA, USA, November 2019. IEEE Computer Society.
[10] Yi Ren, et al.; FastSpeech: Fast, robust and controllable text to speech. In H. Wallach, H. Larochelle, A. Beygelzimer, F. d'Alché-Buc, E. Fox, and R. Garnett, editors, Advances in Neural Information Processing Systems, volume 32. Curran Associates, Inc., 2019.
[11] Alec Radford, et al.; Robust speech recognition via large-scale weak supervision. In Andreas Krause, Emma Brunskill, Kyunghyun Cho, Barbara Engelhardt, Sivan Sabato, and Jonathan Scarlett, editors, Proceedings of the 40th International Conference on Machine Learning, volume 202 of Proceedings of Machine Learning Research, pages 28492-28518. PMLR, 23-29 Jul. 2023.
[12] Hengshuang Zhao, et al.; Point Transformer. In 2021 IEEE/CVF International Conference on Computer Vision (ICCV), pages 16239-16248, 2021.
[13] Meng-Hao Guo, et al.; PCT: Point cloud transformer. Computational Visual Media, 7(2): 187-199, June 2021.
[14] Xumin Yu, et al.; Point-BERT: Pre-training 3D point cloud transformers with masked point modeling. In 2022 IEEE/CVF Conference on Computer Vision and Pattern Recognition (CVPR), pages 19291-19300, 2022.
[15] Daniel Flam-Shepherd, Kevin Zhu, and Alán Aspuru-Guzik. Language models can learn complex molecular distributions. Nature Communications, 13(1): 3293 June 2022.
[16] Carl Edwards, et al.; Translation between molecules and natural language. In Yoav Goldberg, Zornitsa Kozareva, and Yue Zhang, editors, Proceedings of the 2022 Conference on Empirical Methods in Natural Language Processing, pages 375-413, Abu Dhabi, United Arab Emirates, December 2022. Association for Computational Linguistics.
[17] Qizhi Pei, et al.; BioT5: Enriching cross-modal integration in biology with chemical knowledge and natural language associations. In Houda Bouamor, Juan Pino, and Kalika Bali, editors, Proceedings of the 2023 Conference on Empirical Methods in Natural Language Processing, pages 1102-1123, Singapore, December 2023. Association for Computational Linguistics.
[18] Dimitrios Christofidellis, et al.; Unifying molecular and textual representations via multi-task language modelling. In Andreas Krause, Emma Brunskill, Kyunghyun Cho, Barbara Engelhardt, Sivan Sabato, and Jonathan Scarlett, editors, Proceedings of the 40th International Conference on Machine Learning, volume 202 of Proceedings of Machine Learning Research, pages 6140-6157. PMLR, 23-29 Jul. 2023.
[19] Micha Livne, et al.; nach0: multimodal natural and chemical languages foundation model. Chem. Sci., pages -, 2024.
[20] Mario Krenn, et al.; Self-referencing embedded strings (SELFIES): A 100% robust molecular string representation. Machine Learning: Science and Technology, 1(4):045024, October 2020.
[21] Ross Irwin, et al.; Chemformer: a pretrained transformer for computational chemistry. Machine Learning: Science and Technology, 3(1):015022, January 2022.
[22] Daniel Flam-Shepherd and Alán Aspuru-Guzik. Language models can generate molecules, materials, and protein binding sites directly in three dimensions as XYZ, CIF, and PDB files, 2023.
[23] Noel M. O'Boyle, et al.; Open Babel: An open chemical toolbox. Journal of Cheminformatics, 3(1):33, October 2011.
[24] Seyone Chithrananda, et al.; ChemBERTa: large-scale self-supervised pretraining for molecular property prediction. arXiv preprint arXiv:2010.09885, 2020.
[25] Youwei Liang, et al.; Drugchat: Towards enabling chatgptlike capabilities on drug molecule graphs. TechRxiv, 2023.
[26] Shengjie Luo, et al.; One transformer can understand both 2d & 3d molecular data. In The Eleventh International Conference on Learning Representations, 2022.
[27] Xiangru Tang, et al.; Mollm: A unified language model to integrate biomedical text with 2d and 3d molecular representations. Bioinformatics, 2024.
[28] Jascha Sohl-Dickstein, et al.; Deep unsupervised learning using nonequilibrium thermodynamics. In Francis Bach and David Blei, editors, Proceedings of the 32nd International Conference on Machine Learning, volume 37 of Proceedings of Machine Learning Research, pages 2256-2265, Lille, France, 7-9 Jul. 2015. PMLR.
[29] Jonathan Ho, Ajay Jain, and Pieter Abbeel. Denoising diffusion probabilistic models. In H. Larochelle, M. Ranzato, R. Hadsell, M. F. Balcan, and H. Lin, editors, Advances in Neural Information Processing Systems, volume 33, pages 6840-6851. Curran Associates, Inc., 2020.
[30] Emiel Hoogeboom, et al.; Equivariant diffusion for molecule generation in 3D. In Kamalika Chaudhuri, Stefanie Jegelka, Le Song, Csaba Szepesvari, Gang Niu, and Sivan Sabato, editors, Proceedings of the 39th International Conference on Machine Learning, volume 162 of Proceedings of Machine Learning Research, pages 8867-8887. PMLR, 17-23 Jul. 2022.
[31] Xingang Peng, et al.; MolDiff: Addressing the atom-bond inconsistency problem in 3D molecule diffusion generation. In Andreas Krause, Emma Brunskill, Kyunghyun Cho, Barbara Engelhardt, Sivan Sabato, and Jonathan Scarlett, editors, Proceedings of the 40th International Conference on Machine Learning, volume 202 of Proceedings of Machine Learning Research, pages 27611-27629. PMLR, 23-29 Jul. 2023.
[32] Lei Huang, et al.; MDM: Molecular diffusion model for 3D molecule generation. Proceedings of the AAAI Conference on Artificial Intelligence, 37(4):5105-5112 June 2023.
[33] Kristof Schütt, et al.; SchNet: A continuous-filter convolutional neural network for modeling quantum interactions. In I. Guyon, U. Von Luxburg, S. Bengio, H. Wallach, R. Fergus, S. Vishwanathan, and R. Garnett, editors, Advances in Neural Information Processing Systems, volume 30. Curran Associates, Inc., 2017. Minkai Xu, Lantao Yu, Yang Song, Chence Shi, Stefano Ermon, and Jian Tang. GeoDiff: A geometric diffusion model for molecular conformation generation. In International Conference on Learning Representations, 2022.
[34] Bowen Jing, et al.; Torsional diffusion for molecular conformer generation. In S. Koyejo, S. Mohamed, A. Agarwal, D. Belgrave, K. Cho, and A. Oh, editors, Advances in Neural Information Processing Systems, volume 35, pages 24240-24253. Curran Associates, Inc., 2022.
[35] Octavian Ganea, et al.; GeoMol: Torsional geometric generation of molecular 3d conformer ensembles. In M. Ranzato, A. Beygelzimer, Y. Dauphin, P. S. Liang, and J. Wortman Vaughan, editors, Advances in Neural Information Processing Systems, volume 34, pages 13757-13769. Curran Associates, Inc., 2021.
[36] Ilia Igashov, et al.; Equivariant 3d-conditional diffusion model for molecular linker design. Nature Machine Intelligence, April 2024.
[37] Jiaqi Guan, et al.; LinkerNet: Fragment poses and linker co-design with 3D equivariant diffusion. In A. Oh, T. Neumann, Globerson, K. Saenko, M. Hardt, and S. Levine, editors, Advances in Neural Information Processing Systems, volume 36, pages 77503-77519. Curran Associates, Inc., 2023.
[38] Ziqi Chen, et al.; Shape-conditioned 3D molecule generation via equivariant diffusion models. In NeurIPS 2023 Generative AI and Biology (GenBio) Workshop, 2023.
[39] Haitao Lin, et al.; Functional-group-based diffusion for pocket-specific molecule generation and elaboration. In A. Oh, T. Neumann, A. Globerson, K. Saenko, M. Hardt, and S. Levine, editors, Advances in Neural Information Processing Systems, volume 36, pages 34603-34626. Curran Associates, Inc., 2023.
[40] Jiaqi Guan, et al.; 3D equivariant diffusion for target-aware molecule generation and affinity prediction. In The Eleventh International Conference on Learning Representations, 2023.
[41] Jiaqi Guan, et al.; DecompDiff: Diffusion models with decomposed priors for structurebased drug design. In Andreas Krause, Emma Brunskill, Kyunghyun Cho, Barbara Engelhardt, Sivan Sabato, and Jonathan Scarlett, editors, Proceedings of the 40th International Conference on Machine Learning, volume 202 of Proceedings of Machine Learning Research, pages 11827-11846. PMLR, 23-29 Jul. 2023.
[42] Ashish Vaswani, et al.; Attention is all you need. In I. Guyon, U. Von Luxburg, S. Bengio, H. Wallach, R. Fergus, S. Vishwanathan, and R. Garnett, editors, Advances in Neural Information Processing Systems, volume 30. Curran Associates, Inc., 2017.
[43] Jörg Degen, et al.; On the art of compiling and using ‘drug-like’ chemical fragment spaces. Chem Med Chem, 3(10):1503-1507, 2008.
[44] Simon Axelrod and Rafael Gómez-Bombarelli. GEOM, energy-annotated molecular conformations for property prediction and molecular generation. Scientific Data, 9(1):185, April 2022.
[45] Hugo Touvron, et al.; LLaMa: Open and efficient foundation language models, 2023.
[46] Fergus Imrie, et al.; Deep generative models for 3D linker design. Journal of Chemical Information and Modeling, 60(4):1983-1995, 2020. PMID: 32195587.
[47] Yinan Huang, et al.; 3DLinker: An e(3) equivariant variational autoencoder for molecular linker design. In Kamalika Chaudhuri, Stefanie Jegelka, Le Song, Csaba Szepesvari, Gang Niu, and Sivan Sabato, editors, Proceedings of the 39th International Conference on Machine Learning, volume 162 of Proceedings of Machine Learning Research, pages 9280-9294. PMLR, 17-23 Jul. 2022.
[48] Zaixi Zhang, et al.; Molecule generation for target protein binding with structural motifs. In The Eleventh International Conference on Learning Representations, 2023.
[49] Keir Adams and Connor W. Coley. Equivariant shape-conditioned generation of 3D molecules for ligand-based drug design. In The Eleventh International Conference on Learning Representations, 2023.
[50] Daniil Polykovskiy, et al.; Molecular Sets (MOSES): A benchmarking platform for molecular generation models. Frontiers in Pharmacology, 11, 2020.
[51] Shitong Luo, et al.; A 3D generative model for structure-based drug design. In M. Ranzato, et al. editors; Processing Systems, volume 34, pages 6229-6239. Curran Associates, Inc., 2021.
[52] Xingang Peng, et al.; Pocket2Mol: Efficient molecular sampling based on 3D protein pockets. In Kamalika Chaudhuri, Stefanie Jegelka, Le Song, Csaba Szepesvari, Gang Niu, and Sivan Sabato, editors, Proceedings of the 39th International Conference on Machine Learning, volume 162 of Proceedings of Machine Learning Research, pages 17644-17655. PMLR, 17-23 Jul. 2022.
[53] Peter Ertl and Ansgar Schuffenhauer. Estimation of synthetic accessibility score of drug-like molecules based on molecular complexity and fragment contributions. Journal of Cheminformatics, 1(1):8, June 2009.
[54] Amr Alhossary, et al.; Fast, accurate, and reliable molecular docking with Quick Vina 2. Bioinformatics, 31(13):2214-2216, February 2015.
[55] Giovanni Bolcato, et al.; On the value of using 3D shape and electrostatic similarities in deep generative methods. Journal of Chemical Information and Modeling, 62(6):1388-1398, 2022. PMID: 35271260.
[56] Luyao Liu, et al.; TR-Net: a transformer-based neural network for point cloud processing. Machines, 10(7):517, 2022.
[57] Matthew Ragoza, et al.; Generating 3D molecules conditional on receptor binding sites with deep generative models. Chem. Sci., 13:2701-2713, 2022.
[58] Pedro O. Pinheiro, et al.; 3D molecule generation by denoising voxel grids. CoRR, abs/2306.07473, 2023.
[59] Hyung Won Chung, et al.; Scaling instruction-finetuned language models, 2022.
[60] Alexandre Lacoste, et al.; Quantifying the carbon emissions of machine learning. arXiv preprint arXiv:1910.09700, 2019.

1. A transformer model architecture comprising:
a point cloud input module;
a text input module;
a point cloud encoder module operatively coupled to the point cloud input module;
a large language model module operatively coupled to the text input module and point cloud encoder module and configured to receive data therefrom; and
a text output module operatively coupled to the large language model module and configured to output molecular data in line notation format.

2. The transformer model architecture of claim 1, wherein the point cloud encoder module aggregates spatial position information from a point cloud.

3. The transformer model architecture of claim 1, wherein the point cloud encoder module comprises a graph neural network configured to process relative distances between points of a point cloud.

4. The transformer model architecture of claim 3, further comprising a plurality of graph neural network layers.

5. The transformer model architecture of claim 4, wherein each graph neural network layer aggregates information from connected nodes and edges and processes global information from a whole graph.

6. The transformer model architecture of claim 3, wherein the graph neural network comprises attention mechanisms to:
compute attention biases related to edge features and relative positions between points of a point cloud; and
update point embeddings based on the computed attention biases.

7. The transformer model architecture of claim 1, wherein the point cloud encoder module is trained in an unsupervised manner using 3D molecular data.

8. The transformer model architecture of claim 1, wherein the point cloud input module is configured to receive 3D molecular data comprising a point cloud.

9. The transformer model architecture of claim 8, wherein the 3D molecular data comprises, for each point of the point cloud, spatial position data and data representing one or more molecular features.

10. The transformer model architecture of claim 9, wherein the one or more molecular features comprise one or more of the following: an atom symbol, an atom charge, an atom name, or a corresponding amino acid name.

11. The transformer model architecture of claim 8, wherein the 3D molecular data comprises data in at least one of the following chemical language formats: a Simplified Molecular-Input Line Entry System (SMILES) format, a Self-referencing Embedded Strings (SELFIES) format, or a XYZ format.

12. The transformer model architecture of claim 8, wherein the 3D molecular data represents a large ligand or a protein pocket structure.

13. The transformer model architecture of claim 8, wherein the 3D molecular data is down sampled based on one or more prioritized points of the point cloud.

14. The transformer model architecture of claim 13, wherein the one or more prioritized points of the point cloud comprise at least one of the following: a ligand, an alpha carbon (C-alpha) atom, or a terminal atom of a protein amino acid.

15. The transformer model architecture of claim 1, wherein the point cloud encoder module is configured to:
infer a point description based on one or more points in a neighborhood of a point; and
determine a three-dimensional (3D) position of the point relative to the one or more points in the neighborhood of the point.

16. The transformer model architecture of claim 15, wherein the point and the one or more points in the neighborhood of the point are not connected by a direct edge.

17. The transformer model architecture of claim 1, wherein the large language model module and the point cloud encoder module are combined into a single model.

18. A method of pretraining a transformer model, the method comprising:
providing a transformer model having a point cloud input module, a text input module, a point cloud encoder module operatively coupled with the point cloud input module, a large language model module operatively coupled with, and configured to receive data from, the text input module and point cloud encoder module, and a text output module configured to output molecular data in line notation format;
feeding 3D molecular data comprising a point cloud into the point cloud encoder module in an unsupervised manner;
inferring, by the point cloud encoder module, a point description for a point based on one or more points in a neighborhood of the point;
determining a 3D position of the point relative to the one or more points in the neighborhood of the point;
masking or blurring at least some of the one or more points;
predicting, by the point cloud encoder module, at least some of the masked or blurred point features;
sampling random points; and
predicting, by the point cloud encoder module, distances between the random points.

19. The method of claim 18, further comprising:
determining a mask or blur loss value based on the prediction of the masked or blurred point features;
determining a distance loss value based on the prediction of the distances between the random points; and
minimizing a weighted sum of mask or blur loss and distance loss based on the mask loss and distance loss values, respectively.

20. The method of claim 18, further comprising:
encoding the point cloud into one or more point embeddings;
preparing embeddings of an input text sequence;
combining one or more point of point embeddings and the input text sequence embeddings to obtain a fused input; and
feeding the fused input into the point cloud encoder module.

21. A method of shape-conditioned generation, comprising:
training a transformer model to recover a molecule of a molecular point cloud from a blurred region of 3D space where the molecule is located, wherein some parts of a molecular point cloud are blurred and a selected portion of the molecule is unaltered and not blurred, wherein the transformer model architecture comprises:
a point cloud input module;
a text input module;
a point cloud encoder module operatively coupled with the point cloud input module;
a large language model module operatively coupled with, and configured to receive data from, the text input module and point cloud encoder module; and
a text output module configured to output molecular data in line notation format;
inputting, via the point cloud input module, 3D molecular data comprising a point cloud;
inputting, via the text input module, text representing a desired molecule;
processing, by the point cloud encoder and large language model modules, the 3D molecular data and text using the trained point cloud encoder module; and
outputting, by the text output module, a text representing a molecule in line notation.

22. A method of linker design generation, comprising:
training a transformer model to recover a removed part of a molecule when the transformer model does not receive a spatial description of the removed part of the molecule, wherein the transformer model architecture comprises:
a point cloud input module;
a text input module;
a point cloud encoder module operatively coupled with the point cloud input module;
a large language model module operatively coupled with, and configured to receive data from, the text input module and point cloud encoder module; and
a text output module configured to output molecular data in line notation format;
inputting data representing one or more molecules into the trained transformer model;
using the trained transformer model to generate one or more options for a linker portion of a molecule to replace the removed part of the molecule; and
obtaining one or more molecules having the generated one or more options for the linker portion of the molecule.

23. One or more non-transitory computer readable media storing instructions that in response to being executed by one or more processors, cause a computer system to perform operations, the operations comprising:
providing a transformer model having a point cloud input module; a text input module; a point cloud encoder module operatively coupled with the point cloud input module; a large language model module operatively coupled with, and configured to receive data from, the text input module and point cloud encoder module; and a text output module configured to output molecular data in line notation format;
pretraining the point cloud encoder module in an unsupervised manner using 3D molecular data comprising a point cloud;
inferring, by the pretrained point cloud encoder module, a point description based on one or more points in a neighborhood of the point;
determining a 3D position of the point relative to the one or more points in the neighborhood of the point;
masking or blurring at least some of the one or more points;
predicting, by the pretrained point cloud encoder module, at least some of the masked or blurred point features;
sampling random points; and
predicting, by the pretrained point cloud encoder module, distances between the random points.

24. The computer readable media of claim 23, wherein the operations further comprise:
operating the transformer model or pretraining thereof or implementation thereof for shape-conditioned generation or linker design generation of at least one molecule.

25. A computer system comprising:
one or more processors; and
one or more non-transitory computer readable media storing instructions that in response to being executed by the one or more processors, cause the computer system to perform operations, the operations to:
pretrain the point cloud encoder module in an unsupervised manner using 3D molecular data comprising a point cloud;
infer, by the pretrained point cloud encoder module, a point description based on one or more points in a neighborhood of the point;
determine a 3D position of the point relative to the one or more points in the neighborhood of the point;
mask or blur at least some of the one or more points;
predict, by the pretrained point cloud encoder module, at least some of the masked or blurred point features;
sample random points; and
predict, by the pretrained point cloud encoder module, distances between the random points.

26. The computer system of claim 18, further comprising operations to:
operate the transformer model or pretraining thereof or implementation thereof for shape-conditioned generation or linker design generation of at least one molecule.
